(19) United States
(12) Patent Application Publication
(10) Pub. No.: US 2012/0251831 A1
(43) Pub. Date: Oct. 4, 2012

(54) NEAR-INFRARED ABSORPTIVE COLORING MATTER AND NEAR-INFRARED ABSORPTIVE COMPOSITION

(75) Inventors: Akinori Okayasu, Shibukawa-shi (JP); Masaaki Tamura, Shibukawa-shi (JP)

(73) Assignee: JAPAN CARLIT CO., LTD, TOKYO (JP)

(21) Appl. No.: 13/515,698

(22) PCT Filed: Dec. 16, 2010
(86) PCT No.: PCT/JP10/72603

§ 371 Date: Jun. 13, 2012

(30) Foreign Application Priority Data
Dec. 16, 2009 (JP) 2009-285262

(51) Int. Cl. C07C 251/30; C07F 5/02; C07F 9/92; G02B 5/22; B32B 27/40; C08J 3/28; B32B 27/32; C09D 133/00; C09D 123/00; C09D 177/00; C09D 175/04; C07F 9/06; B32B 27/34

(52) U.S. Cl. 428/423.1; 552/301; 552/294; 252/587; 428/474.4; 428/523; 522/153; 522/157; 522/164; 522/162

(57) ABSTRACT

An object of the present invention is to provide a near infrared absorbent dye capable of providing a near infrared shielding filter with excellent transparency as well as high heat resistance and hygrothermal resistance.

The near infrared absorbent dye is characterized in that it is made of the amorphous form of diimmonium salt represented by formula (1).

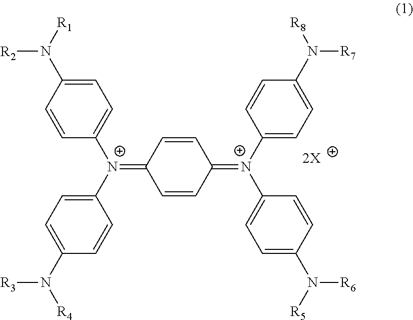

### TECHNICAL FIELD

The present invention pertains to a near infrared absorbent dye made of an amorphous form of diimmonium salt and a near infrared absorptive adhesive composition using the dye, as well as a near infrared absorptive resin composition for hard coat. More specifically, the present invention pertains to a near infrared absorptive composition with excellent transparency in the visible light region and with excellent near infrared absorptive effect, as well as with high heat resistance and hygrothermal resistance, and a near infrared shielding filter using the composition.

### BACKGROUND ART

In recent years, there are increasing demands for display having an increased size and a reduced thickness, and plasma display panels (hereinafter, abbreviated to “PDPs”) are generally spreading widely. A PDP emits near infrared rays and causes an electronic device using a near infrared remote control to malfunction, and therefore it is necessary to intercept near infrared rays with a filter using a near infrared absorbent. In addition, because optical semiconductor elements used in CCD cameras or the like have a high sensitivity in the near infrared region, it is necessary to remove near infrared rays. Near infrared absorbents exhibit the effect of absorbing the heat ray of sunlight, and thus they are used as an heat ray shielding film for automobile glass, building glass and the like. It is also necessary to cut off heat ray in order to prevent a decrease of the output power of solar cell modules caused by a rise in temperature. The near infrared shielding filters for use in these applications are required to effectively absorb rays in the near infrared region while transmitting rays in the visible light region and further have high heat resistance and hygrothermal resistance.

As near infrared absorbent dyes absorbing near infrared rays, conventionally, cyanine dyes, polymethine dyes, squarylium dyes, porphyrin dyes, metal dithiol complex dyes, phthalocyanine dyes, diimmonium dyes, inorganic oxide particles, and the like have been used (Patent Documents 1 and 2).

The near infrared shielding filter for use in PDP contains electromagnetic wave shielding layers, anti-reflection layers, hard coat layers and the like, in addition to the conventional near infrared absorptive layer. Consequently, the near infrared shielding filter for PDP is usually prepared by laminating a near infrared absorptive film, an electromagnetic wave shielding film and an anti-reflection film on a support such as a glass substrate and an impact absorbing material. The near infrared shielding filter for PDP is placed on the front side of the PDP, and it is directly bonded on the support such as a glass substrate and an impact absorbing material for use.

In recent years, in order to form thinner near infrared shielding filters and to simplify the production process of the near infrared shielding filter, there have been attempts to integrate a near infrared absorptive layer and an adhesive layer by using an adhesive containing a near infrared absorbent dye (Patent Document 3).

However, the compounds used as the near infrared absorbent dye, including cyanine dyes, polymethine dyes, squarylium dyes, porphyrin dyes, metal dithiol complex dyes, phthalocyanine dyes, diimmonium dyes and the like, have poor solubility in low-polarity solvents and low-polarity resins. In particular, adhesives are usually of low-polarity and when mixed with a near infrared absorbent dye having a similar polarity, the dye is deposited over time, thereby impairing the appearance and transparency of the coated film.

Also, unlike a binder resin for coating made of polyester resin, acrylic resin or other polymer substance, adhesives have a particular problem in containing a near infrared absorbent dye as typified by a diimmonium dye. That is, the dye degrades significantly after a heat resistance test or a hygrothermal resistance test, resulting in impairment of the near infrared absorptivity. Consequently, extensive studies have been carried out to improve the stability of near infrared absorbent dyes in the adhesive layer.

Patent Document 4 describes an attempt to improve the stability of the near infrared absorbent dye by incorporating the dye and a swelling type layered clay mineral into the adhesive layer. However, the method is disadvantageous in that the swelling type layered clay mineral impairs transparency of the film.

Patent Document 5 discloses an infrared absorptive film using a resin containing micro particles of diimmonium salt as a near infrared absorbent dye, which is believed to be effective for adhesive compositions unable to dissolve dyes because of poor polarity of the resin. However, the method is disadvantageous in that scattering of light generated by the micro particles of the diimmonium salt impairs transparency of the film. In addition, coloring of the diimmonium salt causes a problem of decreasing of the near infrared absorption efficiency.

On the other hand, as explained above, in the conventional optical filter's structure, the near infrared absorptive layer and also the hard coat layer are arranged individually. For example, since the near infrared absorptive layer disclosed in Patent Document 6 does not have a hard coat property, it is necessary to arrange a hard coat layer separately in order to obtain an optical filter with a high scratch resistance. In consideration of this problem, there have been attempts to provide a hard coat layer containing a near infrared absorbent dye, because a hard coat layer having near infrared absorptivity allows the production process to be simplified while using less film

For example, Patent Document 7 discloses a near infrared absorptive resin molded product in which a hard coat layer is formed by coating on at least one side of a transparent resin layer, and the layer contains two or more types of near infrared absorbents including at least one type selected from immonium compound, diimmonium compound, and aminium compound.

Here, the hard coat layer is usually formed by irradiating UV light or other active energy radiation on a hard coat resin. Consequently, when a near infrared absorbent is contained in the hard coat layer, the absorbent is also irradiated by the active energy radiation. It has been found that the conventional diimmonium salt compound disclosed in Patent Document 7 can be easily decomposed by UV light, leading to a significant reduction in the near infrared absorptivity. There is also the problem that curing of the resin is inhibited as a result of the side reaction between the polymerization initializing agent or other curing accelerating agent and the diimmonium salt compound.

Also, Patent Document 8 discloses a resin composition for a hard coat containing a phthalocyanine compound or a naphthalocyanine compound as a near infrared absorbent. However, for the phthalocyanine compound and naphthalocyanine compound, the near infrared absorption region is narrow, and therefore a number of different types of near infrared absorbent dyes with different absorptive wavelengths have to be used to realize a sufficient near infrared absorptivity.

### RELATED ART DOCUMENTS

### Patent Documents

Patent Document 1: JP-A-2003-96040
Patent Document 2: JP-A-2000-80071
Patent Document 3: Japanese Patent No. 3621322
Patent Document 4: JP-A-2008-058472
Patent Document 5: Japanese Patent No. 3987240
Patent Document 6: JP-A-2004-309655
Patent Document 7: Japanese Patent No. 3788652
Patent Document 8: JP-A-2008-268267

### SUMMARY OF THE INVENTION

### Problems to be Solved by the Invention

Consequently, there is a high demand for development of a near infrared absorptive composition that contains a near infrared absorbent dye and can form an adhesive layer and a hard coat layer with excellent transparency, and high heat resistance and hygrothermal resistance. The purpose of the present invention is to provide such a near infrared absorptive adhesive composition and a near infrared absorptive resin composition for hard coat, as well as a near infrared shielding filter using them.

### Means for Solving the Problems

The present inventors have conducted extensive and intensive studies with a view toward solving the above-mentioned problems. As a result, it was found that a near infrared absorptive adhesive composition with excellent transparency by incorporating a near infrared absorbent dye being an amorphous form of diimmonium salt into the adhesive, and that a near infrared shielding filter using the near infrared absorptive adhesive composition has excellent transparency, and high heat resistance and hygrothermal resistance.

In addition, it was found that the amorphous form of diimmonium salt contained in the active energy radiation curable resin can avoid decomposition of the diimmonium salt even when irradiated with a UV light or other active energy radiation, and it can form a hard coat layer with excellent transparency, and high heat resistance and hygrothermal resistance, thus establishing the present invention.

Specifically, the present invention is directed to the followings.

The first invention provides a near infrared absorbent dye being the amorphous form of diimmonium salt represented by formula (1).

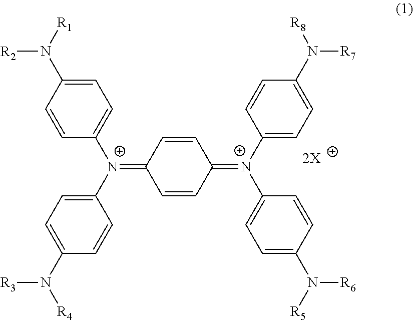

(wherein, R₁ to R₈ are the same or different and each represents an organic group, and X⁻ represents an anion).

The second invention pertains to the near infrared absorbent dye as described in the first invention characterized in that the organic groups R₁ to R₈ in formula (1) represent one selected from the group consisting of n-propyl group, n-butyl group, n-pentyl group, n-hexyl group, and cyclohexyl methyl group.

The third invention pertains to the near infrared absorbent dye as described in the first invention characterized in that the organic groups R₁ to R₈ in formula (1) are two or more types of organic groups, and they are at least two or more types of organic groups selected from the group consisting of n-propyl group, n-butyl group, n-pentyl group, n-hexyl group, and cyclohexyl methyl group.

The fourth invention pertains to the near infrared absorbent dye as described in the first invention characterized in that the organic groups R₁ to R₈ in formula (1) are two different types of organic groups, and, among the two types of organic groups, one type is cyclohexyl methyl group, and the other type is a type of the organic group selected from the group consisting of n-propyl group, n-butyl group, n-pentyl group, and n-hexyl group.

The fifth invention pertains to the near infrared absorbent dye as described in the fourth invention characterized in that the two organic groups in each amino group in formula (1) are a combination of two different types of organic groups.

The sixth invention pertains to the near infrared absorbent dye as described in any of the first through fifth inventions characterized in that X⁻ in formula (1) represents a type selected from the group consisting of hexafluoro phosphate ion, tetrafluoro borate ion, hexafluoro antimonate ion, bis(trifluoromethane sulfonyl) imidate ion, and bis(fluorosulfonyl) imidate iron.

The seventh invention pertains to the near infrared absorbent dye described in any of the first through sixth inventions characterized in that the amorphous form of diimmonium salt represented by formula (1) is prepared by dry pulverizing the crystalline solid of the diimmonium salt.

The eighth invention provides a near infrared absorptive adhesive composition characterized in that the near infrared absorbent dye as described in any of the first through seventh inventions is contained in the solid state in the adhesive.

The ninth invention provides a near infrared shielding filter characterized in that it contains an adhesive layer formed from the near infrared absorptive adhesive composition as described in the eighth invention.

The tenth invention provides a near infrared absorptive resin composition for hard coat characterized in that the near infrared absorbent dye as described in any of the first through seventh inventions is contained in a solid state in the active energy radiation curable resin.

The 11^{th} invention pertains to the resin composition for hard coat as described in the tenth invention characterized in that the active energy radiation curable resin is at least one type of resin selected from the group consisting of polyester-based resin, acrylic-based resin, polyamide-based resin, polyurethane-based resin, and polyolefin-based resin.

The 12^{th} invention provides a near infrared absorptive hard coat material containing a hard coat layer formed by curing the resin composition for hard coat as described in the tenth or 11^{th} invention by means of irradiation of active energy radiation.

The 13^{th} invention pertains to the near infrared absorptive hard coat material as described in the 12^{th} invention characterized in that the hard coat layer is formed on at least one surface of a transparent substrate.

The 14^{th} invention pertains to the near infrared absorptive hard coat material as described in the 13^{th} invention characterized in that the transparent substrate is at least one type selected from the group consisting of glass, PET film, TAC film and electromagnetic wave shielding film.

The 15^{th} invention provides a near infrared shielding filter characterized in that it uses the near infrared absorptive hard coat material as described in any of the 12^{th} through 14^{th} inventions.

### Effects of the Invention

Since the near infrared absorbent dye being an amorphous form of diimmonium salt according to the present invention can exist with high stability in the adhesive and thereby exhibits high heat resistance and hygrothermal resistance, as well as excellent transparency, it can form a near infrared shielding filter with high durability and excellent transparency.

In addition, since the near infrared absorbent dye being an amorphous form of diimmonium salt can avoid decomposition under irradiation of UV light or other active energy radiation, can present with a high stability in the active energy radiation curable resin and has a high transparency, it can form a hard coat layer with excellent near infrared absorptivity, high durability and excellent transparency.

### EMBODIMENT OF THE INVENTION

The near infrared absorbent dye being an amorphous form of diimmonium salt according to the present invention will be explained below.

### [Near Infrared Absorbent Dye]

The near infrared absorbent dye of the present invention is characterized in that it is made of an amorphous form of diimmonium salt. In this invention, near infrared radiation refers to light with a wavelength in the range of 750-2000 nm.

The diimmonium salt used in the present invention is represented by formula (1) (hereinafter to be referred to as “diimmonium salt (1)”.

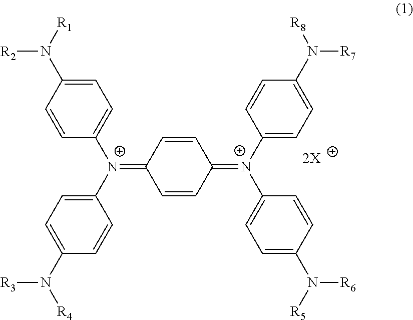

In formula (1), R₁ to R₈ are the same or different and each represents an organic group, and X⁻ represents an anion.

Preferred examples of the organic groups represented by R₁ to R₈ include a linear or branched C₁₋₁₀ alkyl group optionally substituted with halogen atoms, a C₃₋₁₂ cycloalkyl group, and a C₃₋₁₂ cycloalkyl-C₁₋₁₀ alkyl group in which the cycloalkyl ring is optionally substituted.

Examples of the linear or branched C₁₋₁₀ alkyl group include a methyl group, an ethyl group, a n-propyl group, a n-butyl group, a n-pentyl group, a n-hexyl group, an iso-propyl group, an iso-butyl group, a sec-butyl group, a tert-butyl group, a n-amyl group, an iso-amyl group, a 1-methyl butyl group, a 2-methyl butyl group, a 1-ethyl butyl group, a 2-ethyl butyl group, a 2-dimethyl propyl group, and a 1,1-dimethyl propyl group. Of these, a n-propyl group, a n-butyl group, a n-pentyl group, and a n-hexyl group are preferred as they can lead to a lower crystallinity of diimmonium salt (1), thereby facilitating the amorphous formation. In addition, such alkyl groups with a low polarity are preferred because mixing with the adhesive is facilitated owing to their similar polarities.

Examples of the C₃₋₁₂ cycloalkyl group include a cyclopentyl group and a cyclohexyl group.

In the C₃-C₁₂ cycloalkyl-C₁-C₁₀ alkyl group, the cycloalkyl ring may be substituted or unsubstituted, and examples of substituents include an alkyl group, a hydroxyl group, a sulfonic acid group, an alkylsulfonic acid group, a nitro group, an amino group, an alkoxy group, a halogenoalkyl group, and a halogen atom. The cycloalkyl ring is preferably unsubstituted, and a cycloalkyl-alkyl group represented by formula (2) is preferred because of its low solubility in the acrylic-based resin and the like used for the adhesive or the resin for hard coat.

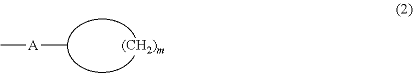

In formula (2), A represents a linear or branched alkyl group having 1 to 10 carbon atoms, and m represents an integer of 3 to 12.

A preferably has 1 to 4 carbon atoms, and m is preferably 5 to 8, especially preferably 5 to 6.

Examples of the cycloalkyl-alkyl group represented by formula (2) include a cyclopentyl methyl group, a 2-cyclopentyl ethyl group, a 2-cyclopentyl propyl group, a 3-cyclopentyl propyl group, a 4-cyclopentyl butyl group, a 2-cyclohexyl methyl group, a 2-cyclohexyl ethyl group, a 3-cyclohexyl propyl group, a 4-cyclohexyl butyl group. Of these, a cyclopentyl methyl group, a cyclohexyl methyl group, a 2-cyclohexyl ethyl group, a 2-cyclohexyl propyl group, a 3-cyclohexyl propyl group, and a 4-cyclohexyl butyl group are preferred, and a cyclopentyl methyl group and a cyclohexyl methyl group are more preferred. A cyclohexyl methyl group is especially preferred because its low solubility in the acrylic-based resin and the like used for the adhesive or the resin for hard coat leads to a lower polarity.

Examples of the linear or branched C₁₋₁₀ alkyl group optionally substituted by halogen atoms include a 2-halogeno ethyl group, a 2,2-dihalogeno ethyl group, a 2,2,2-trihalogeno ethyl group, a 3-halogeno propyl group, a 3,3-dihalogeno propyl group, a 3,3,3-trihalogeno propyl group, a 4-halogeno butyl group, a 4,4-dihalogeno butyl group, a 4,4,4-trihalogeno butyl group, a 5-halogeno pentyl group, a 5,5-dihalogeno pentyl group, a 5,5,5-trifluoro pentyl group, and other halogenated alkyl groups. Of these, mono-halogenated alkyl groups represented by formula (3) are preferred.

—CₙH₂ₙ—CH₂Y (3)

In formula (3), n represents an integer of 1 to 9, and Y represents a halogen atom.

n is preferably 1 to 9, and more preferably 1 to 4. Especially preferably, Y is a fluorine atom. Specifically, examples include a 2-fluoro ethyl group, a 3-fluoro propyl group, a 4-fluoro butyl group, a 5-fluoro pentyl group, and other mono-fluoro alkyl groups.

R₁ to R₈ in formula (1) may all be of the same type of organic group, or they may be two or more different types of organic groups. It is preferred that they be two different types of organic groups. Especially, the following combinations of two different types of organic groups are preferred: R₁ and R₂, R₃ and R₄, R₅ and R₆, and R₇ and R₈. That means diimmonium salt (1) in which two organic groups in each amino group are a combination of two different types of organic groups is preferred.

It is preferred that the two types of organic groups be organic groups selected from the group consisting of n-propyl group, n-butyl group, n-pentyl group, n-hexyl group, and cyclohexyl methyl group. In addition, it is more preferred that one type of organic group be a cyclohexyl methyl group, while the other type of organic group be selected from the group consisting of n-propyl group, n-butyl group, n-pentyl group and n-hexyl group.

As the organic groups of one amino group are a combination of two different types of organic groups, the crystallinity of diimmonium salt (1) can be decreased, and the amorphous formation is facilitated. Especially, if one type among the two types of organic groups is a cyclohexyl methyl group, decrease in the crystallinity due to the steric hindrance contribute more to the amorphous formation.

In formula (1), X⁻ represents an anion needed for neutralizing the charge in the diimmonium cations. It may be made of organic acid anions, inorganic anions, or the like.

Examples of the anions include a fluorine ion, a chlorine ion, a bromine ion, an iodine ion, and other halogen ions, a perchlorate ion, a periodate ion, a tetrafluoro borate ion, a hexafluoro phosphate ion, a hexafluoro antimonite ion, a bis(trifluoro methane sulfonyl) imidate ion, and a bis(fluoro sulfonyl) imidate ion.

Of these, the especially preferred types include a tetrafluoro borate ion, a hexafluoro phosphate ion, a hexafluoro antimonate ion, a bis(trifluoro methane sulfonyl) imidate ion, and a bis(fluoro sulfonyl) imidate ion. They can provide the near infrared shielding filter with high heat resistance and hygrothermal resistance. A hexafluoro phosphate ion, a hexafluoro antimonate ion, and a bis(fluoro sulfonyl) imidate ion are especially preferred because these ions are highly inorganic and therefore the obtained diimmonium salt has a low solubility in the acrylic-based resin and the like used for the adhesive and the resin for hard coat.

The following diimmonium salts represented by formula (1) used in the present invention are preferred as they have high heat resistance and hygrothermal resistance, and excellent transparency:
N,N,N′,N′-tetrakis{p-di(cyclohexylmethyl)aminophenyl}-p-phenyl ene diimmonium hexafluoro phosphate,
N,N,N′,N′-tetrakis{p-di(cyclohexylmethyl)aminophenyl}-p-phenyl ene diimmonium hexafluoro antimonate,
N,N,N′,N′-tetrakis{p-di(cyclohexylmethyl)aminophenyl}-p-phenyl ene diimmonium bis(trifluoro methane sulfonyl) imidate,
N,N,N′,N′-tetrakis{p-di(cyclohexylmethyl)aminophenyl}-p-phenyl ene diimmonium bis(fluoro sulfonyl) imidate,
N,N,N′,N′-tetrakis{p-di(n-propyl)aminophenyl}-p-phenylene diimmonium hexafluoro phosphate,
N,N,N′,N′-tetrakis{p-di(n-propyl)aminophenyl}-p-phenylene diimmonium hexafluoro antimonate,
N,N,N′,N′-tetrakis{p-di(n-propyl)aminophenyl}-p-phenylene diimmonium bis(trifluoro methane sulfonyl)imidate,
N,N,N′,N′-tetrakis{p-di(n-propyl)aminophenyl}-p-phenylene diimmonium bis(fluoro sulfonyl)imidate,
N,N,N′,N′-tetrakis{p-di(n-butyl)aminophenyl}-p-phenylene diimmonium hexafluoro phosphate,
N,N,N′,N′-tetrakis{p-di(n-butyl)aminophenyl}-p-phenylene diimmonium hexafluoro antimonate,
N,N,N′,N′-tetrakis{p-di(n-butyl)aminophenyl}-p-phenylene diimmonium bis(trifluoro methane sulfonyl)imidate,
N,N,N′,N′-tetrakis{p-di(n-butyl)aminophenyl}-p-phenylene diimmonium bis(fluoro sulfonyl)imidate,
N,N,N′,N′-tetrakis{p-di(n-pentyl)aminophenyl}-p-phenylene diimmonium hexafluoro antimonate,
N,N,N′,N′-tetrakis{p-di(n-pentyl)aminophenyl}-p-phenylene diimmonium bis(fluoro sulfonyl)imidate,
N,N,N′,N′-tetrakis{p-(cyclohexylmethyl-n-propyl)aminophenyl}-p-phenylene diimmonium hexafluoro phosphate,
N,N,N′,N′-tetrakis{p-(cyclohexylmethyl-n-propyl)aminophenyl}-p-phenylene diimmonium bis(fluoro sulfonyl) imidate,
N,N,N′,N′-tetrakis{p-(cyclohexylmethyl-n-butyl)aminophenyl}-p-phenylene diimmonium hexafluoro phosphate,
N,N,N′,N′-tetrakis{p-(cyclohexylmethyl-n-butyl)aminophenyl}-p-phenylene diimmonium bis(fluoro sulfonyl)imidate,
N,N,N′,N′-tetrakis{p-(cyclohexylmethyl-n-pentyl)aminophenyl}-p-phenylene diimmonium hexafluoro phosphate,
N,N,N′,N′-tetrakis{p-(cyclohexylmethyl-n-pentyl)aminophenyl}-p-phenylene diimmonium bis(fluoro sulfonyl)imidate and the like.
   Especially, the following types are more preferred as they have low crystallinity, thereby facilitating the amorphous formation:
N,N,N′,N′-tetrakis{p-di(cyclohexylmethyl)aminophenyl}-p-phenyl ene diimmonium hexafluoro phosphate,
N,N,N′,N′-tetrakis{p-di(cyclohexylmethyl)aminophenyl}-p-phenyl ene diimmonium bis(trifluoro methane sulfonyl) imidate,
N,N,N′,N′-tetrakis{p-di(cyclohexylmethyl)aminophenyl}-p-phenyl ene diimmonium bis(fluoro sulfonyl) imidate,
N,N,N′,N′-tetrakis{p-di(n-propyl)aminophenyl}-p-phenylene diimmonium hexafluoro phosphate,
N,N,N′,N′-tetrakis{p-di(n-butyl)aminophenyl}-p-phenylene diimmonium hexafluoro antimonate,
N,N,N′,N′-tetrakis{p-di(n-butyl)aminophenyl}-p-phenylene diimmonium bis(fluoro sulfonyl)imidate,
N,N,N′,N′-tetrakis{p-di(n-pentyl)aminophenyl}-p-phenylene diimmonium hexafluoro antimonate,
N,N,N′,N′-tetrakis{p-(cyclohexylmethyl-n-propyl)aminophenyl}-p-phenylene diimmonium hexafluoro phosphate,
N,N,N′,N′-tetrakis{p-(cyclohexylmethyl-n-butyl)aminophenyl}-p-phenylene diimmonium hexafluoro phosphate and the like.

In the present specification, the amorphous form refers to a solid in which the atoms or molecules are not arranged in a regular periodic configuration to form a crystal. As far as the crystallinity of the solid is concerned, it is determined by measuring the diffraction pattern on a powder X-ray diffraction device. Here, for the amorphous form, the diffraction pattern determined on the powder X-ray diffraction device displays a pattern without detection of diffraction peaks that would indicate the crystallinity. For example, the diffraction peaks are measured on the powder X-ray diffraction device, and for the peak detected with the highest intensity, the peak top is taken from the baseline, and the full width at half maximum 2θ is determined. For the amorphous form here, this value is 1° or larger. Such solid substantially does not contain crystal, and it is made of only the amorphous form.

The near infrared absorbent dye of the present invention can be prepared from the amorphous formation of the crystalline solid of diimmonium salt (1) by dry pulverizing. Here, “dry pulverizing” means a pulverizing operation without using a solvent. Here, “pulverizing” refers to a treatment in which a mechanical pressure is applied to destroy the crystal structure. Usually, pulverizing can be carried out using a pulverizing device that applies a pressure to crush the crystal, such as a ball mill, a sand mill, a paint shaker, an attritor, a hammer mill, a roll mill, a kneader, an extruder, or an automatic mortar. As needed, a pulverizing medium, such as glass beads, steel beads, zirconia beads, or alumina beads, may be used. In addition, a roller compactor or other dry compressing granulating machine may be used.

By means of the dry pulverizing treatment, the crystalline solid of diimmonium salt (1) loses the crystallinity, and it becomes an amorphous form.

The dry pulverizing is carried out until the crystallinity of diimmonium salt (1) disappears. That is, it is carried out until no clear diffraction peak can be detected from the diffraction pattern on the powder X-ray diffraction device. More specifically, for example, the diffraction peaks are measured on the powder X-ray diffraction device, and for the peak with the highest intensity detected, the full width at half maximum 2θ is then determined with reference to the distance to the peak top from the baseline, and the dry pulverizing operation is carried out until this full width at half maximum becomes over 1°.

By containing the amorphous form thus prepared as a near infrared absorbent dye, a near infrared absorptive adhesive composition and a near infrared shielding filter using the composition can have higher heat resistance and hygrothermal resistance, and better transparency as compared with those containing the dye in a crystalline state, such as crystal or aggregates of crystals.

The near infrared absorbent dye made of the amorphous form of diimmonium salt (1) prepared as mentioned above can be used after mixing with any solvent. Here, “mixing” refers to the operation in that a powder is agitated in the presence of a solvent and mixed up in the solvent. It does not include “wet pulverizing”. Here, “wet pulverizing” refers to the operation of pulverizing in the presence of a solvent, and a pulverizing medium, such as glass beads, steel beads, zirconia beads, or alumina beads, may be used as needed. Although “wet pulverizing” may be carried out to convert the crystalline solid of diimmonium salt (1) to the amorphous form, it is difficult to apply a pressure on the crystalline solid, and it takes a long time for the amorphous formation in the wet pulverizing operation. In addition, since the wet pulverizing operation is subject to the formation of aggregates and has a particular problem of re-coagulation caused by over dispersion, it would be hard to obtain the near infrared absorbent dye of the present invention.

Examples of the solvents that can be used in the mixing operation include methanol, ethanol, propanol, isopropanol, butanol, and other alcohol-based solvents; ethylene glycol, propylene glycol, butylene glycol, polyethylene glycol, polypropylene glycol, polyoxyethylene polyoxypropylene copolymer, and other glycol-based solvents; monomethyl ether, monoethyl ether, monopropyl ether, monoisopropyl ether, monobutyl ether and the like of the glycol-based solvents, and other ether alcohol-based solvents; dimethyl ether, diethyl ether, dipropyl ether, diisopropyl ether, dibutyl ether, methyl ethyl ether, methyl propyl ether, methyl isopropyl ether, methyl butyl ether, ethyl propyl ether, ethyl isopropyl ether, ethyl butyl ether and the like of the glycol-based solvents, and other polyether-based solvents; methyl ethyl ketone, methyl isobutyl ketone, cyclohexanone, and other ketone-based solvents; methyl acetate, ethyl acetate, butyl acetate, and other ester-based solvents; and hexane, heptane, octane, cyclopentane, cyclohexane, toluene, xylene, and other hydrocarbon-based solvents. These solvents may be used either alone (1 type) or as a mixture of two or more types. Among them, the organic solvents with a boiling point of 200° C. or lower are preferred from the standpoint of enhancing the coating workability with the near infrared absorptive composition. In such a solvent, 0.01-80 mass %, preferably 0.5-50 mass % of the near infrared absorbent dye made of the amorphous form of diimmonium salt (1) is added and agitated, thereby providing a mixture with diimmonium salt (1) dispersed in solid state of amorphous form.

The crystalline solid of diimmonium salt (1) can be prepared using the following method.

That is, in a polar solvent such as N-methyl-2-pyrrolidone (hereinafter to be referred to as NMP) or dimethyl formamide (hereinafter to be referred to as DMF), the amino substance represented by formula (4) and obtained in the Ullmann reaction and reducing reaction is reacted with iodide corresponding to R₁ to R₈ and carbonate of alkyl metal as de-iodination agent at 30-150° C., preferably at 70-120° C., to form a diimmonium salt precursor represented by formula (5). For example, when R₁ to R₈ are all cyclohexyl methyl groups, iodinated cyclohexyl alkane as the corresponding iodide is added. On the other hand, when R₁ to R₈ are two or more different types of organic groups, iodides in mole numbers corresponding to the respective organic groups are used for successive reactions just as mentioned previously, or they may be added simultaneously. For example, when R₁ to R₈ are cyclohexyl methyl group and other organic groups, iodinated cyclohexyl alkane in mole number corresponding to the number of the substituent groups is added and after reaction, the iodides in mole numbers corresponding to the numbers of substituent groups (such as iodinated fluoro alkane, iodinated alkane, alkoxy iodine, iodinated benzene, iodinated benzyl, iodinated phenethyl, and other phenyl-1-iodide alkanes) are added sequentially, or these different types of iodides may be added simultaneously.

To prepare diimmonium salt (1) in which the two substituents of each amino group are a combination of two different types of organic groups, an imine substance represented by formula (6) is obtained by reaction of an amino substance represented by formula (4) with alkyl aldehyde compounds corresponding to R₁, R₃, R₅ and R₇ in toluene, and then a secondary amine substance represented by formula (7) is obtained by reducing reaction in a hydrogen atmosphere by means of a palladium carbon catalyst. Further reaction of alkyl iodides corresponding to R₂, R₄, R₆ and R₈ is carried out in the same way as above to give the diimmonium salt precursor represented by formula (5), which has combinations of R₁ and R₂, R₃ and R₄, R₅ and R₆, and R₇ and R₈ each as a combination of two different types of organic groups.

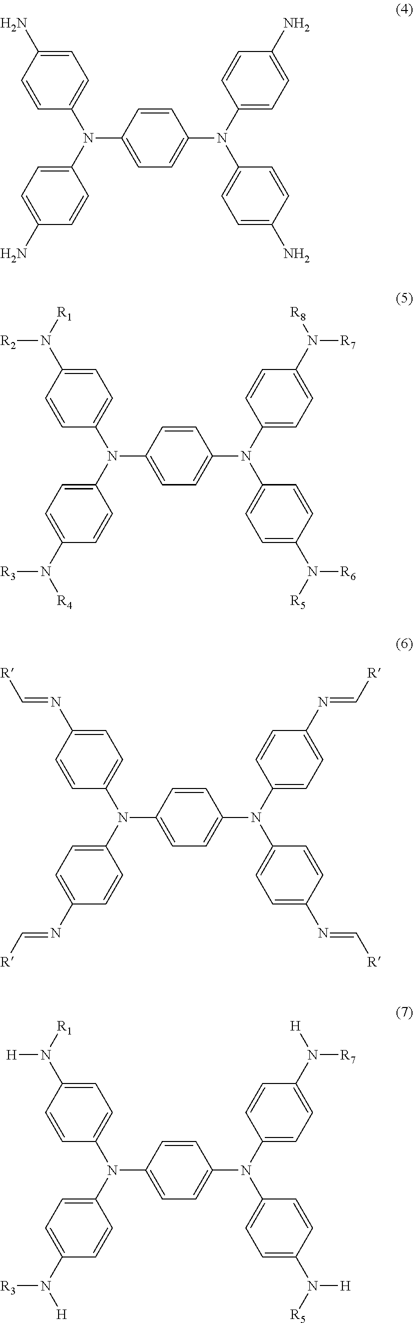

R₁ to R₈ are just as defined above.

Then, in an organic solvent such as NMP, DMF, or acetonitrile, the diimmonium salt represented by formula (5) is reacted with the silver salt of the corresponding anion X⁻ at a temperature in the range of 30-150° C., preferably in the range of 40-80° C. After the deposited silver is filtered off, a solvent such as water, ethyl acrylate, or hexane, is added, and the generated precipitate is filtered to give a crystalline solid of the diimmonium salt represented by formula (1).

By dry pulverizing the crystalline solid of diimmonium salt (1) prepared as above, the amorphous form of diimmonium salt (1) can be obtained.

For example, in an automatic mortar AMN-200 (product of Nitto Kagaku Co., Ltd.), the crystalline solid of diimmonium salt obtained as above is put in a 150 mm agate mortar, followed by dry pulverizing with a rotation velocity of 100 rpm for the mortar rod and 6 rpm for the mortar container for about 10-120 minutes. As a result, the amorphous form of diimmonium salt is obtained. The pulverized substance thus obtained is measured for its diffraction peaks on a powder X-ray diffraction device (RINT2200 manufactured by Rigaku Corporation) using a CuKα ray as the X-ray source, with tube voltage of 40 kV, tube current of 20 mA, scanning range (2θ) in the range of 3-60°, spread slit of ½°, scattering slit of ½°, light receiving slit of 0.15 mm, sampling width of 0.02°, and scanning speed of 4°/min. Pulverizing is carried out until no clear diffraction peak can be detected. For example, pulverizing may be carried out until the full width at half maximum 2θ of the peak with respect to the distance from the baseline to the peak top becomes 1° or larger.

### Adhesive Composition

### (Adhesive)

There is no specific restriction on the type of the adhesive for preparing the near infrared absorptive adhesive composition of the present invention, as long as it can form a transparent layer on the surface of a transparent substrate, and the function of the optical filter is not impaired. Examples of the adhesives include acrylic-, polyester-, polyamide-, polyurethane-, polyolefin-, polycarbonate-, rubber- and silicone-based adhesives. Of these, acrylic-based adhesives are preferred as they have excellent transparency, adhesiveness, heat resistance and the like.

Preferred examples of the acrylic-based adhesives include those containing acrylic-based polymers that have acrylate or methacrylate having C₁₋₁₄ alkyl groups as the main ingredient, such as methyl (meth)acrylate, ethyl (meth)acrylate, n-butyl (meth)acrylate, t-butyl (meth)acrylate, isobutyl (meth)acrylate, hexyl (meth)acrylate, 2-ethyl hexyl (meth)acrylate, and n-octyl (meth)acrylate.

An acrylic-based polymer may be appropriately crosslinked to obtain an adhesive sheet with high heat resistance. As the specific means of crosslinking, one may use a so-called crosslinking agent, a compound having groups that can react with a hydroxyl group, an amino group, an amido group and the like contained as appropriate crosslinking base points in the acrylic-based polymer, such as a polyisocyanate compound, an epoxy compound, an aziridine compound and the like. Especially, a polyisocyanate compound and an epoxy compound are preferred.

As well as excellent adhesiveness and coagulating ability, the aforementioned acrylic-based adhesive has a high stability with respect to light and oxygen owing to the absence of unsaturated bond in the polymer. The adhesive is preferred also because of its high degree of selectivity of the monomer type and molecular weight. In terms of securing adhesion to the transparent supporting film, a high molecular weight (polymerization degree) is preferred. Specifically, the weight-average molecular weight (Mw) of the principal polymer is in the range of 600,000-2,000,000, preferably in the range of 800,000-1,800,000.

There is no specific restriction on the proportion of the near infrared absorbent dye to the adhesive in the near infrared absorptive adhesive composition of the present invention. The proportion can be adjusted to ensure the desired properties, especially high efficiency in near infrared absorptivity, excellent transparency in the visible light region, and high heat resistance and hygrothermal resistance. For example, when an adhesive layer of 10-30 μm is formed, the preferable content of the near infrared absorbent dye with respect to 100 parts by mass of the solid content of the adhesive is in the range of 0.01-50 parts by mass, more preferably in the range of 0.1-20 parts by mass, and most preferably in the range of 1-10 parts by mass. If the content is less than 0.01 part by mass, it is hard to realize an excellent near infrared absorptivity. On the other hand, if the content exceeds 50 parts by mass, improvement of the aforementioned properties may level off despite an increase in the content, which is undesirable in consideration of the cost, and the transparency in the visible light region may be lost. The proportion of the near infrared absorbent dye of the present invention can be adjusted depending on the desired transmissivity of the adhesive in the visible light and near infrared region as well as the thickness of the adhesive layer. In addition, as needed, another one or several types of near infrared absorbent dyes may be used at the same time. The proportion of the other types of near infrared absorbent dyes to 100 parts by mass of the solid content of the adhesive may be in the range of 0.01-10 parts by mass. As long as the effect of the present invention is not hampered, the other type of near infrared absorbent dyes may be dissolved in the adhesive, or dispersed in the solid state of micro particles, aggregates or the like. Diimmonium salt (1) in such states other than the amorphous form may also be contained.

### (Solvent)

The near infrared absorptive adhesive composition of the present invention may also contain a solvent. Use of a solvent is preferred from the standpoint of enhancing the coating workability in application of the adhesive composition.

There is no specific restriction on the type of the solvent. Examples of the solvents include methanol, ethanol, propanol, isopropanol, butanol, and other alcohol-based solvents; ethylene glycol, propylene glycol, butylene glycol, polyethylene glycol, polypropylene glycol, polyoxyethylene polyoxypropylene copolymer, and other glycol-based solvents; monomethyl ether, monoethyl ether, monopropyl ether, monoisopropyl ether, monobutyl ether and the like of the glycol-based solvents, and other ether alcohol-based solvents; dimethyl ether, diethyl ether, dipropyl ether, diisopropyl ether, dibutyl ether, methyl ethyl ether, methyl propyl ether, methyl isopropyl ether, methyl butyl ether, ethyl propyl ether, ethyl isopropyl ether, ethyl butyl ether and the like of the glycol-based solvents, and other polyether-based solvents; methyl ethyl ketone, methyl isobutyl ketone, cyclohexanone, and other ketone-based solvents; methyl acetate, ethyl acetate, butyl acetate, and other ester-based solvents; and hexane, heptane, octane, cyclopentane, cyclohexane, toluene, xylene, and other hydrocarbon-based solvents.

These solvents may be used either alone or as a mixture of two or more types. An organic solvent with a boiling point of 200° C. or lower is preferred. The water content in the solvent is preferably 5 mass % or less.

The content of the solvent in the near infrared absorptive adhesive composition of the present invention is in the range of 20-90 mass %, preferably in the range of 50-80 mass %.

### (Additives)

The near infrared absorptive adhesive composition of the present invention may also contain appropriate additives as needed. Examples of the additives include a curing agent, a curing accelerating agent, an adhesiveness imparting agent, a viscosity modifier, a leveling agent, a dripping inhibitor, a pigment, a pigment dispersing agent, a surfactant, a UV light absorbent, a photosensitizing agent, an oxidation inhibitor, a lightproof stabilizer, a corrosion inhibitor, a rust inhibitor, a peroxide decomposing agent, a filler, a reinforcing agent, a plasticizer, a lubricant, an emulsifier, a fluorescent whitening agent, an organic flame inhibitor, an inorganic flame inhibitor, an antistatic agent, a defoaming agent, a silane coupling agent, and an anti-blocking agent.

The near infrared absorptive adhesive composition of the present invention may also contain any appropriate organic or inorganic micro particles. Usually, the organic or inorganic micro particles are used for imparting the desired functions including adjustment of refractive index, electroconductive property, and the like.

Examples of the micro particles used for imparting a higher refractive index or electroconductive property of the layer made of the adhesive composition include zinc oxide, titanium oxide, zirconium oxide, aluminum oxide, tin oxide, tin doped indium oxide, antimony doped tin oxide, indium doped zinc oxide, indium oxide, and antimony oxide. Examples of the micro particles used for decreasing the refractive index of the layer made of the adhesive composition include magnesium fluoride, silica, and silica balloons. These micro particles may be used either alone or as a combination of two or more types.

The content of the organic or inorganic micro particles in the near infrared absorptive adhesive composition of the present invention is usually in the range of 0.01-50 mass %, preferably in the range of 0.1-30 mass %.

To prepare the near infrared absorptive adhesive composition of the present invention, the near infrared absorbent dye of the present invention or its mixture with a solvent is added to the adhesive, and, after adding a solvent, a curing agent and the like as needed, they are blended according to a conventional method.

### [Near Infrared Shielding Filter]

The near infrared shielding filter of the present invention has a structure containing a transparent substrate and an adhesive layer. It is preferably designed to ensure that the transmissivity of near infrared radiation at a wavelength of 800-1100 nm is 20% or lower.

### (Transparent Substrate)

As the transparent substrate, sheet-, film-, or plate-shaped transparent substrates may be used. Examples of the materials for making the transparent substrate include polyethylene terephthalate (PET), polyethylene naphthalate (PEN), and other polyester resins; triacetyl cellulose (TAC); methyl methacrylate-based copolymer, and other acrylic resins; styrene resin, polysulfone resin, polyether sulfone resin, polycarbonate resin, polyvinyl chloride resin, polymethacryl imide resin, and glass.

A transparent substrate treated for easier bonding may also be used. For example, the PET film may be a film treated for easier bonding (easy-bondable PET film). The easy-bonding treatment is preferably carried out at least on the surface of the side where the adhesive layer is formed. Examples of the methods for the easy-bonding treatment include a treatment to form an easy-bonding layer and a treatment of the surface of the substrate by corona processing. An example of an easy-bonding layer is an easy-bonding resin layer or the like.

Especially preferable transparent substrates include glass, polyethylene terephthalate (PET) film, and triacetyl cellulose (TAC) film.

To prepare the near infrared shielding filter of the present invention, the transparent substrate is coated with the near infrared absorptive adhesive composition of the present invention, followed by drying.

The methods of coating of the adhesive composition include a flow coating method, a spray method, a bar coating method, a gravure coating method, a roll coating method, a blade coating method, an air knife coating method, a lip coating method, a die coater method, and other known coating methods. The coating is carried out in such a way that the thickness of film is in the range of 5-50 μm, preferably in the range of 10-30 μm, and the resultant film is firmed as an adhesive layer by drying at a temperature in the range of 80-140° C., preferably in the range of 100-130° C. Usually, ageing treatment is carried out after drying. The condition of the ageing treatment depends on the type of the resin and the type of the crosslinking agent to be used, and for the near infrared absorptive adhesive composition of the present invention, storage is preferably in a thermostatically controlled vessel at 25-50° C. for about 1 day-1 week.

The near infrared shielding filter of the present invention prepared using the adhesive composition of the present invention as explained above has at least an adhesive layer of the adhesive composition formed on a transparent substrate, and may have further layers of a transparent substrate, a glass sheet, a filter and the like with other functions as needed.

### (Resin Composition for Hard Coat)

The resin composition for hard coat of the present invention contains the near infrared absorbent dye of the present invention and a resin as the base material. A polymerization initiator and any other ingredients may be included as needed.

### [Active Energy Radiation Curable Resin (Hard Coat Resin Ingredient)]

There is no specific restriction on the base resin of the resin composition for hard coat, and any type may be used as long as it has good transparency and active energy radiation curability. The hard coat resin ingredient has active energy radiation curability. Specifically, the hard coat resin ingredient is an active energy radiation curable resin that can be cured under irradiation of active energy. In addition, there is no restriction on the type of active energy radiation. Examples of the active energy radiation include electron beam, UV light, visible light, and infrared radiation. Because a higher energy of the radiation can facilitate curing, preferable active energy radiations include UV light and electron beam, and particularly UV light.

In consideration of the active energy radiation curability, the preferable hard coat resin ingredient is radical polymeric resin. There is no specific restriction on the type of the radical polymeric resin. Usually, a radical polymeric resin having two or more carbon-carbon double bonds in each molecule is preferred. Preferable types of radical polymeric resins include polyester-based resins, (meth)acrylic-based resins, polyamide-based resins, polyurethane-based resins, and polyolefin-based resins. An energy radiation curable radical polymeric resin other than the aforementioned radical polymeric resin having two or more carbon-carbon double bonds in each molecule may also be used as a reactive diluent or the like without deviating from the purpose of the present invention.

The aforementioned (meth)acrylic-based resin refers to a (meth)acrylic-based polymer prepared by polymerization of (meth)acrylate as monomer. The (meth)acrylic-based polymer may be prepared by polymerization of one type of (meth)acrylate as monomer, or by polymerization of two or more types of (meth)acrylates as monomers, or by polymerization of (meth)acrylate and a compound that can be copolymerized with (meth)acrylate (hereinafter to be referred to as “copolymerizable compound”) as monomers. Examples of the (meth)acrylates that may be used as the monomer include methyl (meth)acrylate, ethyl (meth)acrylate, propyl (meth)acrylate, butyl (meth)acrylate, pentyl (meth)acrylate, hexyl (meth)acrylate, octyl (meth)acrylate, nonyl (meth)acrylate, decyl (meth)acrylate, dodecyl (meth)acrylate, 2-ethyl hexyl (meth)acrylate, and other C₁₋₂₀ alkyl (meth)acrylates and their substituted forms; 2-hydroxy ethyl (meth)acrylate, 2-hydroxy butyl (meth)acrylate, 2-hydroxy propyl (meth)acrylate, 2-hydroxy-3-phenoxy propyl (meth)acrylate, and other (meth)acrylates containing hydroxyl groups; (meth)acrylic acid, and other (meth)acryl containing carboxyl groups; phenyl (meth)acrylate, benzyl (meth)acrylate, and other aryl (meth)acrylates; methoxy ethyl (meth)acrylate, ethoxy ethyl (meth)acrylate, butoxy ethyl (meth)acrylate, ethoxy propyl (meth)acrylate, and other alkoxy alkyl (meth)acrylates; ethoxy diethylene glycol (meth)acrylate, phenoxy polyethylene glycol (meth)acrylate, nonyl phenol ethylene oxide (EO) adduct (meth)acrylate, nonyl phenol propylene oxide (PO) adduct (meth)acrylate, and other alcohol's oxy alkylene adduct's (meth)acrylates; and cyclohexyl (meth)acrylate, and other cycloalkyl (meth)acrylates. In addition to the above-listed compounds, other (meth)acrylates may also be used. The (meth)acrylates may be used either alone or as a mixture of two or more types. Examples of the compounds that can be used as monomer for copolymerization include the compounds having ethylenic unsaturated bonds. The compound having ethylenic unsaturated bonds refers to a compound obtained by substituting hydrogen atoms in ethylene (CH₂═CH₂). As long as it is possible to perform copolymerization with the (meth)acrylate without hampering the effect of the present invention, other compounds may also be used as monomer. Examples of the other copolymerizable compounds include styrene, vinyl toluene, α-methyl styrene, vinyl naphthalene, halogenated styrene, and other aromatic vinyl monomers; vinyl acetate, and other vinyl ester monomers; vinyl chloride, vinylidene chloride, and other halogenated vinyl monomers; (meth)acrylamide, N-methylol (meth)acrylamide, N-methoxy methyl (meth)acrylamide, N-butoxy methyl (meth)acrylamide, N,N-dimethyl acrylamide, and other amido group-containing vinyl monomers; (meth)acrylonitrile, and other nitrile group-containing monomers; and vinyl ether-based monomers.

### (Near Infrared Absorbent Dye)

There is no specific restriction on the proportion of the near infrared absorbent dye of the present invention to the hard coat resin ingredient in the composition for hard coat of the present invention. This proportion can be adjusted to realize the desired properties, especially a high efficiency of near infrared absorptivity, excellent transparency in the visible light region, and high heat resistance and hygrothermal resistance. For example, 0.01-50 parts by mass, more preferably 0.1-30 parts by mass, and most preferably 1-20 parts by mass of the near infrared absorbent dye of the present invention is added to 100 parts by mass of the solid content of the hard coat resin. If the content is less than 0.01 part by mass, it is hard to realize excellent near infrared absorptivity. On the contrary, if the content exceeds 50 parts by mass, improvement of the aforementioned properties may level off despite an increase in the content, which is undesirable in consideration of the cost, and the transparency in the visible light region may be lost. The proportion of the near infrared absorbent dye of the present invention can be adjusted depending on the desired transmissivity in the visible light and near infrared region as well as the thickness of the hard coat layer. In addition, another one or several types of near infrared absorbent dyes may be used at the same time as needed. The content of the other types of near infrared absorbent dyes with respect to 100 parts by mass of the solid content of the hard coat resin ingredient is in the range of 0.01-20 parts by mass. As long as the effect of the present invention is not hampered, other near infrared absorbent dyes may be dissolved in the hard coat resin ingredient, or dispersed in the solid state of micro particles, aggregates, or the like. Diimmonium salt (1) in such a state other than the amorphous form may also be used.

### (Polymerization Initiator)

It is preferred that the resin composition for hard coat of the present invention contain an polymerization initiator. The energy radiation sensitive radical polymerization initiator is a preferred polymerization initiator. Preferred examples include acetophenone-based compounds, benzyl-based compounds, benzophenone-based compounds, thioxanthone-based compounds, and other ketone-based compounds.

Examples of the acetophenone-based compounds include diethoxy acetophenone, 2-hydroxy-2-methyl-1-phenylpropane-1-one, 4′-isopropyl-2-hydroxy-2-methyl propiophenone, 2-hydroxy methyl-2-methyl propiophenone, 2,2-dimethoxy-1,2-diphenyl ethane-1-one, p-dimethyl amino acetophenone, p-tertiary butyl dichloro acetophenone, p-tertiary butyl trichloro acetophenone, p-azidobenzal acetophenone, 1-hydroxy cyclohexyl phenyl ketone, 2-methyl-1-[4-(methyl thio)phenyl]-2-morpholino propanone-1,2-benzyl-2-dimethyl amino-1-(4-morpholino phenyl)-butanone-1, benzoin, benzoin methyl ether, benzoin ethyl ether, benzoin isopropyl ether, benzoin-n-butyl ether, and benzoin isobutyl ether.

Examples of the benzyl-based compounds include benzyl and anicyl.

Examples of the benzophenone-based compounds include benzophenone, methyl o-benzoyl benzoate, Michler's ketone, 4,4′-bisdiethyl amino benzophenone, 4,4′-dichlorobenzophenone, and 4-benzoyl-4′-methyl diphenyl sulfide.

Examples of the thioxanthone-based compounds include thioxanthone, 2-methyl thioxanthone, 2-ethyl thioxanthone, 2-chloro thioxanthone, 2-isopropyl thioxanthone, and 2,4-diethyl thioxanthone.

The polymerization initiators may be used either alone or as a mixture of two or more types depending on the desired properties. The content of the polymerization initiator with respect to the solid content of the hard coat resin is in the range of 0.01-20 mass %, preferably in the range of 0.1-10 mass %. If the content of the polymerization initiator is less than 0.01 mass %, the composition cannot be sufficiently cured. On the contrary, if the content of the polymerization initiator exceeds 20 mass %, the properties of the cured substance level off, and even some adverse influence appears. Besides, the cost effectiveness may become diminished in this case.

### (Solvent)

The resin composition for hard coat of the present invention may also contain a solvent. Use of a solvent is preferred from the standpoint of enhancing the coating workability in application of the resin composition for hard coat. There is no specific restriction on the type of the solvent. Examples of the solvents that may be used include methanol, ethanol, propanol, isopropanol, butanol, and other alcohol-based solvents; ethylene glycol, propylene glycol, butylene glycol, polyethylene glycol, polypropylene glycol, polyoxyethylene polyoxypropylene copolymer, and other glycol-based solvents; monomethyl ether, monoethyl ether, monopropyl ether, monoisopropyl ether, monobutyl ether and the like of the glycol-based solvents, and other ether alcohol-based solvents; dimethyl ether, diethyl ether, dipropyl ether, diisopropyl ether, dibutyl ether, methyl ethyl ether, methyl propyl ether, methyl isopropyl ether, methyl butyl ether, ethyl propyl ether, ethyl isopropyl ether, ethyl butyl ether and the like of the glycol-based solvents, and other polyether-based solvents; methyl ethyl ketone, methyl isobutyl ketone, cyclohexanone, and other ketone-based solvents; methyl acetate, ethyl acetate, butyl acetate, and other ester-based solvents; and hexane, heptane, octane, cyclopentane, cyclohexane, toluene, xylene, and other hydrocarbon-based solvents. These solvents may be used either alone or as a mixture of two or more types. The organic solvents with a boiling point of 200° C. or lower are preferred. The water content in the solvent is preferably 5 mass % or less. The content of the solvent in the resin composition for hard coat of the present invention is in the range of 10-90 mass %, preferably in the range of 50-80 mass %.

### (Monofunctional Polymeric Compound)

The resin composition for hard coat in the present invention may also contain an appropriate monofunctional polymeric compound as needed. Examples of the monofunctional polymeric compounds include acrylamide, (meth)acryloyl morpholine, 7-amino-3,7-dimethyl octyl (meth)acrylate, isobutoxy methyl (meth)acrylate, isobornyloxy ethyl (meth)acrylate, isobornyl (meth)acrylate, 2-ethyl hexyl (meth)acrylate, ethyl diethylene glycol (meth)acrylate, t-octyl (meth)acrylamide, diacetone (meth) acrylamide, dimethylamino ethyl (meth)acrylate, diethylamino ethyl (meth)acrylate, lauryl (meth)acrylate, dicyclopentadiene (meth)acrylate, dicyclopentenyloxy ethyl (meth)acrylate, N,N-dimethyl (meth)acrylamide, tetrachloro phenyl (meth)acrylate, 2-tetrachlorophenoxy ethyl (meth)acrylate, tetrahydro furfuryl (meth)acrylate, tetrabromo phenyl (meth)acrylate, 2-tetrabromo phenoxy ethyl (meth)acrylate, 2-trichlorophenoxy ethyl (meth)acrylate, tribromophenyl (meth)acrylate, 2-tribromo phenoxy ethyl (meth)acrylate, 2-hydroxy ethyl (meth)acrylate, 2-hydroxy propyl (meth)acrylate, vinyl caprolactam, N-vinyl pyrrolidone phenoxy ethyl (meth)acrylate, butoxy ethyl (meth)acrylate, pentachloro phenyl (meth)acrylate, pentabromophenyl (meth)acrylate, polyethylene glycol mono-(meth)acrylate, polypropylene glycol mono-(meth)acrylate, bornyl (meth)acrylate, and methyl triethylene diglycol (meth)acrylate.

### (Additives)

The resin composition for hard coat of the present invention may also contain appropriate additives as needed. Examples of the additives include a leveling agent, a pigment, a pigment dispersing agent, a UV light absorbent, an oxidation inhibitor, a viscosity modifier, a lightproof stabilizer, a metal inactivating agent, a peroxide decomposing agent, a filler, a reinforcing agent, a plasticizer, a lubricant, a corrosion inhibitor, a rust inhibitor, an emulsifier, a demolding agent, a fluorescent whitening agent, an organic flame inhibitor, an inorganic flame inhibitor, a dripping inhibitor, a melt flow modifier, an antistatic agent, a sliding improving agent, a close contact improving agent, a soiling inhibitor, a surfactant, a defoaming agent, a polymerization inhibitor, a photosensitizing agent, a surface improving agent, and a silane coupling agent. When a UV light absorbent is used, of course, the amount should be small enough to avoid inhibition of the curing reaction of the hard coat resin ingredient.

The resin composition for hard coat of the present invention may also contain any appropriate organic or inorganic micro particles. Usually, the organic or inorganic micro particles are used for imparting the desired functions (adjustment of refractive index, electroconductive property, glare preventing property and the like). Examples of the micro particles used for imparting a higher refractive index or electroconductive property of the layer of the resin composition for hard coat include zinc oxide, titanium oxide, zirconium oxide, aluminum oxide, tin oxide, tin doped indium oxide, antimony doped tin oxide, indium doped zinc oxide, indium oxide, and antimony oxide. Examples of the micro particles used for decreasing refractive index of the layer of the resin composition for hard coat include magnesium fluoride, silica, and silica balloons. Examples of the micro particles used for preventing glare include, in addition to the above-listed types of micro particles, calcium carbonate, barium sulfate, talc, kaolin, and other inorganic particles; silicone resin, melamine resin, benzoguanamine resin, acrylic resin, polystyrene resin, and copolymer resins using them, and other organic micro particles. These micro particles may be added either alone or as a combination of two or more types. The content of the organic or inorganic micro particles in the resin composition for hard coat of the present invention is usually in the range of 0.01-50 mass %, preferably in the range of 0.1-30 mass %.

To prepare the resin composition for hard coat of the present invention, the near infrared absorbent dye of the present invention or its mixture with a solvent is added to the hard coat resin ingredient, and, after adding a solvent, a polymerization initiator and the like as needed, they are blended according to a conventional method.

### [Hard Coat Material]

The hard coat material pertaining to the present invention contains the hard coat layer formed of the resin composition for hard coat, and it has near infrared absorptivity. The hard coat material may be only the hard coat layer formed of the resin composition for hard coat, or it may have a hard coat layer and a substrate. The hard coat material may be used in plastic optical members, touch panels, film type liquid crystal elements, plastic moldings, and the like. An example of the substrate contained in the hard coat material is a transparent substrate.

Since the hard coat layer itself in the hard coat material also has near infrared absorptivity, there is no need to form a near infrared absorptive layer separately. When the hard coat layer and the near infrared absorptive layer are formed separately, a PET film or other base film need to be placed between these layers. However, according to the present invention, such base film is not required.

### (Transparent Substrate)

The preferable hard coat material of the present invention has a transparent substrate and a hard coat layer. There is no specific restriction on the type of the transparent substrate. The transparent substrate may have a sheet shape, a film shape or a plate shape. Examples of the materials for making the transparent substrate include polyethylene terephthalate (PET), polyethylene naphthalate (PEN), and other polyester resins; triacetyl cellulose (TAC); methyl methacrylate-based copolymer, and other acrylic resins; styrene resin, polysulfone resin, polyether sulfone resin, polycarbonate resin, polyvinyl chloride resin, polymethacryl imide resin, and glass. In addition, compounds having lactone structure may also be used as the material for making the transparent substrate. Especially, the preferable transparent substrates include glass, polyethylene terephthalate (PET) film, triacetyl cellulose (TAC) film, and resin film having a lactone structure.

A transparent substrate treated for easier bonding may also be used. For example, the PET film may be a film treated for easier bonding (easy-bondable PET film). The easy-bonding treatment is preferably carried out at least on the surface of the side where the hard coat layer is formed. Examples of the methods for the easy-bonding treatment include a treatment to form an easy-bonding layer and a treatment of the surface of the substrate by corona processing. An example of an easy-bonding layer is an easy-bonding resin layer or the like.

In addition, an electromagnetic wave shielding film prepared by treatment for shielding electromagnetic waves may be used preferably as the transparent substrate. The electromagnetic wave shielding film is a film that can shield electromagnetic waves to suppress the adverse influence of the electromagnetic waves generated from a display device or the like on a living body and an electronic equipment. The electromagnetic wave shielding film may contain metal and the like that can shield the electromagnetic waves. More preferably, the electromagnetic wave shielding film has an electromagnetic wave shielding layer formed on the surface of a resin film. Examples of the electromagnetic wave shielding layers include thin films and metal mesh layers. Examples of the thin films include thin films made of silver, copper, indium oxide, zinc oxide, indium tin oxide, antimony tin oxide, and other metals and metal oxides. The thin film may be formed using the conventional methods, such as a vacuum vapor deposition method, an ion plating method, a sputtering method, a CVD method, and a plasma chemical vapor deposition method. The metal mesh layer is a metal layer with mesh-like holes formed on it. Examples of the metal mesh layers include metal mesh layers made of copper or silver. The most typical electromagnetic wave shielding layer is the thin film of indium tin oxide (may be abbreviated as ITO). As another electromagnetic wave shielding layer, a laminate prepared by alternately laminating a dielectric layer and a metal layer on a substrate may be used preferably. The dielectric layer is preferably made of indium oxide, zinc oxide, or other transparent metal oxide, and the metal layer is usually made of silver or silver-palladium alloy. The laminate is usually formed by laminating odd-numbered layers (about 3-13 layers) starting with a dielectric layer.

The following are some examples of the electromagnetic wave shielding films: an electromagnetic wave shielding material prepared by vapor depositing a metal or a metal oxide to form a thin film electroconductive layer on a transparent substrate (see JP-A-1-278800 or JP-A-5-323101), an electromagnetic wave shielding material prepared by embedding highly electroconductive fibers in a transparent substrate (see JP-A-5-327274 or JP-A-5-269912), an electromagnetic wave shielding material prepared by directly printing an electroconductive resin containing metal powder or the like on a transparent substrate (see JP-A-62-57297 or JP-A-2-52499), an electromagnetic wave shielding material prepared by forming a transparent resin layer on a transparent substrate, and then forming a copper mesh pattern on it by means of electroless plating method (see JP-A-5-283889), and the like.

The hard coat material of the present invention can be prepared by coating the transparent substrate with the resin composition for hard coat of the present invention to form a resin composition layer, and irradiating the layer with an active energy radiation, such as UV light, electron beam, or the like, thereby curing the layer to form a hard coat layer. Coating with the resin composition may be carried out using any of known methods, including a cast method, a flow coating method, a spray method, a bar coating method, a gravure coating method, a roll coating method, a blade coating method, an air knife coating method, a lip coating method, and a die coater method. The finished film thickness is usually in the range of 0.5-20 μm, preferably in the range of 1-10 μm. When UV light is used as the active energy, the intensity of the UV light with wavelength of about 180-400 nm may be irradiated in the range of 20-200 mJ/cm².

### [Heat Ray Shielding Film]

The heat ray shielding film, an example of the near infrared shielding filter of the present invention, may be formed by coating the transparent substrate with the resin composition for hard coat of the present invention using the cast method or other known method, followed by irradiation with an active energy radiation to form a hard coat layer.

The heat ray shielding film can be formed by only using one type of or two or more types of the near infrared absorbent of the present invention. However, if the near infrared shielding performance is scant near a wavelength of 850 nm, phthalocyanine dyes, dithiol-based metal complex, or other known dyes may be added. Further, in order to improve the lightproof property, benzophenone-based UV light absorbent dyes, benzotriazole-based UV light absorbent dyes, or other UV light absorbent dyes may be added. Also, a known dye that absorbs the visible light region may be add to adjust the tone.

### [Anti-Reflection Film]

The anti-reflection film as an example of the near infrared shielding filter of the present invention has an anti-reflection layer. Usually, the anti-reflection layer is the top layer. The anti-reflection layer forms the surface of the anti-reflection film.

The following are some types of the anti-reflection layer: (1) a layer prepared by alternately laminating a layer made of a high refractive index material and a layer made of a low refractive index material; (2) a layer prepared by sequentially laminating a layer made of a material having an intermediate refractive index between that of the low refractive index material and the high refractive index material, a layer made of the high refractive index material, and a layer made of the low refractive index material; (3) a single layer made of a low refractive index material, and the like. For the anti-reflection layer having plural layers, the “high refractive index”, “intermediate refractive index” and “low refractive index” indicate the relative magnitude relationship among the values of the refractive indexes of the various layers of the anti-reflection layer. More specifically, the anti-reflection layer may have any of the following structures: a single layer made of a low refractive index layer; a 2-layer structure layer prepared by laminating sequentially a high refractive index layer and a low refractive index layer; a 4-layer structure layer prepared by laminating sequentially a high refractive index layer, a low refractive index layer, a high refractive index layer and a low refractive index layer; a 3-layer structure layer prepared by sequentially laminating an intermediate refractive index layer, a high refractive index layer and a low refractive index layer; and the like. Any type of anti-reflection layer may be adopted as long as it is possible to realize a low average reflectivity, excellent anti-reflection performance, and good visibility by means of appropriate optical design.

The anti-reflection layer preferably contains a hard coat layer formed of the resin composition for hard coat of the present invention. In this case, the hard coat layer may also function as an anti-reflection layer. A preferable anti-reflection layer contains a hard coat layer and a layer with refractive index different from that of the hard coat layer (hereinafter to be referred to as layer with different refractive index). The layer with different refractive index may constitute at least one anti-reflection layer. More preferably, the anti-reflection film contains a hard coat layer and a low refractive index layer, which is laminated on the outer side of the hard coat layer and has a refractive index lower than that of the hard coat layer. In this case, the anti-reflection layer is composed of a high refractive index layer made of the hard coat layer and a low refractive index layer laminated on the outer side of the high refractive index layer. Also, the anti-reflection layer may be formed separately from the hard coat layer.

The refractive index of the low refractive index layer is preferably 1.5 or lower from the standpoint of reducing the reflectivity.

Examples of the materials for making the low refractive index layer include MgF₂ (refractive index is about 1.4), SiO₂ (refractive index is about 1.2-1.5), LiF (refractive index is about 1.4), 3NaF.AlF₃ (refractive index is about 1.4), and Na₃AlF₆ (refractive index is about 1.33). The low refractive index layer may be prepared by using a material made by dispersing the micro particles of MgF₂, SiO₂, and the like in a UV light or electron beam curable resin or a silicon alkoxide-based matrix. However, the present invention is not limited to this scheme.

As far as the method for forming the low refractive index layer is concerned, when the matrix containing the low refractive index micro particles described above is used, coating may be performed so as to form a film having a thickness in the range of 0.01-1 μm, and then drying treatment, UV light irradiation treatment, or electron beam irradiation treatment may be carried out as needed.

Coating to form the low refractive index layer may be carried out using any of the known methods, such as a method using rod or wire bar, as well as a micro-gravure method, a gravure method, a die method, a curtain method, a lip method, a slot method, and various other types of coating methods.

In addition, the low refractive index layer may also be formed using any of the following methods: a vacuum vapor deposition method, a sputtering method, a reactive sputtering method, an ion plating method, an electroplating method, and the like.

On the other hand, examples of the materials for making the high refractive index layer include TiO₂ (refractive index is 2.3-2.7), Y₂O₃ (refractive index is 1.9), La₂O₃ (refractive index is 2.0), ZrO₂ (refractive index is 2.1), Al₂O₃ (refractive index is 1.6), Nb₂O₃ (refractive index is 1.9-2.1), In₂O₃ (refractive index is 1.9-2.1), Sn₂O₃ (refractive index is 1.9-2.1), and In—Sn composite oxide (ITO, refractive index is 1.9-2.1). The high refractive index layer may be formed by dispersing micro particles of the TiO₂, Y₂O₃, La₂O₃, ZrO₂, Al₂O₃, Nb₂O₃, In₂O₃, Sn₂O₃, In—Sn composite oxide, and the like in a matrix. In addition to the aforementioned hard coat resin ingredient, the matrix may also be made of other types, such as UV curable resin, electron beam curable resin, and silicon alkoxide-based compound. The resin composition for hard coat containing the micro particles can be used as a high refractive index layer.

To prepare the high refractive index layer using the matrix containing the high refractive index micro particles described above, coating may be performed so as to form a film having a thickness in the range of 0.01-1 μm and then drying treatment, UV light irradiation treatment, or electron beam irradiation treatment may be carried out as needed. The same method as the low refractive index layer may be adopted to perform the coating operation.

The high refractive index layer may also be formed using any of the following methods: a vacuum vapor deposition method, a sputtering method, a reactive sputtering method, an ion plating method, an electroplating method, and the like.

The intermediate refractive index layer may be made of a substance having an intermediate refractive index between the refractive index of the low refractive index material and the refractive index of the high refractive index material. The method for forming the intermediate refractive index layer is the same as for the low refractive index layer or high refractive index layer.

Other functional layers may also be formed in the anti-reflection film. Examples of the functional layers include a soiling inhibiting layer, an antistatic layer, an electromagnetic wave shielding layer, and a neon light correcting layer. These functional layers may be made of the known materials and by using the known methods. One layer may have plural functions. These functional layers may also be used in a glare-preventing film or an optical film for a thin shaped display unit of the present invention. Especially, when it is used for a display unit, the soiling preventing layer is preferably formed near the top, as compared to the anti-reflection layer.

When used in a plasma display unit, the anti-reflection film preferably has an electromagnetic wave shielding layer and/or a neon light correcting layer. There is no specific restriction on the configuration of these layers. Usually, in consideration of the visibility and the like, it is preferred that they be formed on the side opposite to the side where the anti-reflection layer is formed with respect to the substrate. The electromagnetic wave shielding layer or the neon light correcting layer may also be used in the aforementioned glare-preventing film or the optical film for a thin-shaped display unit.

### [Glare-Preventing Film]

The glare-preventing film, an example of the near infrared shielding filter of the present invention, uses the aforementioned hard coat material. For example, the glare-preventing film has a hard coat layer formed of the resin composition for hard coat of the present invention containing micro particles, and a transparent substrate. It can be prepared by coating the transparent substrate with the resin composition for hard coat of the present invention containing micro particles using the cast method or other known method, followed by irradiation with active energy radiation to form a hard coat layer. By adding micro particles, it is possible to impart the glare-preventing property to the layer made of the resin composition for hard coat (hard coat layer), thereby enabling the hard coat layer to also function as a glare-preventing layer. Also, a glare-preventing layer may be formed separately from the hard coat layer.

There is no specific restriction on the micro particles for imparting the glare-preventing property. These micro particles preferably have transparency. Organic or inorganic micro particles may be used and the organic micro particles are preferred. There is no specific restriction on the type of the organic micro particles. For example, plastic beads or the like may be used. Examples of the plastic beads include styrene beads (refractive index is 1.59), melamine beads (refractive index is 1.57), acrylic beads (refractive index is 1.49), acryl-styrene beads (refractive index is 1.54), polycarbonate beads, and polyethylene beads. An example of the inorganic micro particles is silica beads. Also, the organic/inorganic composite micro particles disclosed in JP-A-10-330409 or JP-A-2004-307644 may be used. In order to increase the refractive index of the glare-preventing layer, it is preferred that the oxide of at least one type of metal selected from the group consisting of titanium, zirconium, aluminum, indium, zinc, tin and antimony be used. In this case, an inorganic filler with the mean grain size of 0.2 μm or smaller, preferably 0.1 μm or smaller may be used.

The hard coat layer (glare-preventing layer) may contain a leveling agent, a UV light absorbent, a UV stabilizer, a fluorescent whitening agent, an antistatic agent, a fingerprint attachment inhibitor, and the like. The leveling agent used here may be that commonly used in preparing paint or other coating forming composition.

### [Optical Filter for Thin-Shaped Display Unit]

The optical filter for thin-shaped display unit as an example of the near infrared shielding filter of the present invention uses the aforementioned hard coat material, the aforementioned anti-reflection film or the aforementioned glare-preventing film.

The resin composition for hard coat of the present invention is preferred for the optical filter. In addition to high-efficiency absorption of the near infrared radiation, the optical filter also has a high transparency in the visible light region due to the amorphous form of diimmonium salt (1). For example, the total light ray transmissivity for the visible light region is preferably 40% or higher, more preferably 50% or higher, and the transmissivity for the near infrared radiation with wavelength in the range of 800-1100 nm is preferably 30% or lower, more preferably 15% or lower.

The optical filter may have a hue adjusting layer, a glass or other substituting member in addition to the hard coat layer. For example, the optical filter may be prepared by coating a glass or other supporting material with the resin composition for hard coat of the present invention using the cast method or other known method, followed by irradiation of an active energy radiation to form a hard coat layer.

The structure of the various layers of the optical filter may be selected at will. Preferably, the optical filter has the anti-reflection layer or the glare-preventing layer as the top layer (facing the user). When the various layers are laminated with each other, a physical treatment, such as a corona treatment, plasma treatment, or the like, may be carried out, and a known high-polarity polymer, such as polyethylene imine, oxazoline-based polymer, polyester, cellulose, or the like may also be used as an anchoring coating agent.

An electromagnetic wave shielding layer may be formed separately from the hard coat layer. Examples of the electromagnetic wave shielding layer include thin film and metal mesh layer. Examples of the thin film include thin films made of silver, copper, indium oxide, zinc oxide, indium tin oxide, antimony tin oxide, and other metals and metal oxides. The thin film may be formed using the conventional methods, such as a vacuum vapor deposition method, an ion plating method, a sputtering method, a CVD method, and a plasma chemical vapor deposition method. The metal mesh layer is a metal layer with mesh-like holes formed on it. Examples of the metal mesh layers include metal mesh layers made of copper or silver. The most typical electromagnetic wave shielding layer is the thin film of indium tin oxide (may be abbreviated as ITO). As another electromagnetic wave shielding layer, a laminate prepared by alternately laminating dielectric layer and metal layer on a substrate may be used preferably. The dielectric layer is preferably made of indium oxide, zinc oxide, or other transparent metal oxide. The metal layer is usually made of silver or silver-palladium alloy. The laminate is usually formed by laminating odd-numbered layers (about 3-13 layers) starting with a dielectric layer.

The optical filter for thin-shaped display unit may be arranged away from the display device, or it may be directly bonded on the display device. When it is arranged away from the display device, use of a glass supporting member is preferred. When directly bonded on the display device, an optical filter devoid of glass is preferred.

Conventionally, in a near infrared shielding filter for PDP or the like using a near infrared absorbent dye made of diimmonium salt, the substituent groups are designed to enable the diimmonium to be dissolved in the resin with a view to improving the heat resistance and hygrothermal resistance.

However, even in such near infrared absorbent dye, the solubility in the low-polarity solvent and the low-polarity resin is still poor. Especially, because adhesives usually have a very low polarity, the near infrared absorbent dye added to such adhesives becomes deposited over time, thereby impairing the appearance and transparency of the coated film. In addition, adhesives have a particular problem, which is that the dye degrades significantly after a heat resistance test or a hygrothermal resistance test, resulting in impairment of the near infrared absorptivity.

On the other hand, for the hard coat resin, as it is formed under irradiation of UV light or other active energy radiation, the near infrared absorbent dye is decomposed under UV light, and the near infrared absorptivity degrades significantly. Another problem is that curing of the resin may be inhibited by a side reaction of the diimmonium salt compound with the polymerization initiator or other curing accelerating agent.

Also, an attempt to prevent degradation of the dye by adding the near infrared absorbent dye as micro particles instead of dissolving in the resin leads to scattering of light and impairment of the transparency, which means that the optical characteristics can no longer meet the demands on the filter.

The present invention has been made in consideration of the aforementioned problems, based on the finding that a diimmonium salt contained in the amorphous state in an adhesive composition or hard coat resin composition can provide an adhesive layer or a hard coat layer with high heat resistance and hygrothermal resistance as well as excellent transparency.

### EXAMPLES

Hereinbelow, the present invention will be described in more detail with reference to the following Examples, which should not be construed as limiting the scope of the invention.

### Production Example 1

### Production of N,N,N′,N′-tetrakis{p-di(cyclohexylmethyl)aminophenyl}-p-phenyl ene diimmonium hexafluoro phosphate

To 100 parts of DMF were added 10 parts of N,N,N′,N′-tetrakis{p-aminophenyl}-p-phenylene diamine, 63 parts of cyclohexylmethyl iodide and 30 parts of potassium carbonate, and the resultant mixture was subjected to reaction at 120° C. for 10 hours. The reaction mixture was poured into 500 parts of water, and the resultant precipitate was collected by filtration and washed with 500 parts of methyl alcohol, and then dried at 100° C. to obtain 24.1 parts of N,N,N′,N′-tetrakis{p-di(cyclohexylmethyl)aminophenyl}-p-phenyl ene diamine.

To 24.1 parts of the obtained N,N,N′,N′-tetrakis{p-di(cyclohexylmethyl)aminophenyl}-p-phenyl ene diamine were added 200 parts of acetonitrile and 7.9 parts of silver hexafluorophosphate, and the resultant mixture was subjected to reaction at 60° C. for 3 hours, and the resultant silver was filtered off. Then, 200 parts of water was added to the filtrate, and the resultant precipitate was collected by filtration and dried to obtain 27.0 parts of N,N,N′,N′-tetrakis{p-di(cyclohexylmethyl)aminophenyl}-p-phenyl ene diimmonium hexafluoro phosphate.

### Production Example 2

### Production of N,N,N′,N′-tetrakis{p-di(cyclohexyl methyl)aminophenyl}-p-phenylene diimmonium bis(trifluoro methane sulfonyl) imidate

32 parts of N,N,N′,N′-tetrakis{p-di(cyclohexyl methyl)aminophenyl}-p-phenylene diimmonium bis(trifluoro methane sulfonyl) imidate were obtained in substantially the same manner as in Production Example 1, except that instead of silver hexafluoro phosphate, silver bis(trifluoro methane sulfonyl) imidate was used.

### Production Example 3)

### Production of N,N,N′,N′-tetrakis{p-di(n-propyl)aminophenyl}-p-phenylene diimmonium hexafluoro phosphate

19 parts of N,N,N′,N′-tetrakis{p-di(n-propyl)aminophenyl}-p-phenylene diimmonium hexafluoro phosphate were obtained in substantially the same manner as in Production Example 1, except that instead of cyclohexyl methyl iodide, 1-iodo propane was used.

### Production Example 4

### Production of N,N,N′,N′-tetrakis{p-di(n-butyl)aminophenyl}-p-phenylene diimmonium hexafluoro antimonate

18 parts of N,N,N′,N′-tetrakis{p-di(n-butyl)aminophenyl}-p-phenylene diamine were obtained in substantially the same manner as in Production Example 1, except that instead of cyclohexyl methyl iodide, 1-iodo butane was used.

To 18 parts of the obtained N,N,N′,N′-tetrakis{p-di(n-butyl)aminophenyl}-p-phenylene diamine were added 200 parts of acetonitrile and 12.9 parts of silver hexafluoro antimonate, and the resultant mixture was subjected to reaction at 60° C. for 3 hours, and the resultant silver was filtered off. Then, 200 parts of water was added to the filtrate, and the resultant precipitate was collected by filtration and dried to obtain 24.5 parts of N,N,N′,N′-tetrakis{p-di(n-butyl)aminophenyl}-p-phenylene diimmonium hexafluoro antimonate.

### Production Example 5)

### Production of N,N,N′,N′-tetrakis{p-di(n-pentyl)aminophenyl}-p-phenylene diimmonium hexafluoro antimonate

26.5 parts of N,N,N′,N′-tetrakis{p-di(n-pentyl)aminophenyl}-p-phenylene diimmonium hexafluoro antimonate were obtained in substantially the same manner as in Production Example 4, except that instead of 1-iodo butane, 1-iodo pentane was used.

### Production Example 6

### Production of N,N,N′,N′-tetrakis{p-di(n-butyl)aminophenyl}-p-phenylene diimmonium bis(fluoro sulfonyl)imidate

27.5 parts of N,N,N′,N′-tetrakis{p-di(n-butyl)aminophenyl}-p-phenylene diimmonium bis(fluoro sulfonyl)imidate were obtained in substantially the same manner as in Production Example 4, except that instead of silver hexafluoro antimonate, silver bis(fluoro sulfonyl) imidate was used.

### Production Example 7

### Production of N,N,N′,N′-tetrakis{p-(cyclohexylmethyl-n-propyl)aminophenyl}-p-phenylene diimmonium hexafluoro phosphate

To 100 parts of toluene were added 10 parts of N,N,N′,N′-tetrakis(p-aminophenyl)-p-phenylene diamine and 12 parts of cyclohexane carboxy aldehyde, and the resultant mixture was subjected to reaction at 80° C. for 5 hours. After being cooled to the room temperature, 3 parts of palladium carbon catalyst were added, and hydrogen gas was blown in for 2 hours for hydrogenation reaction. Then, 18 parts of 1-iodo propane and 15 parts of potassium carbonate were added, and reaction was carried out at 120° C. for 6 hours. After filtering of the reaction liquid, the filtrate was added to 500 parts of methyl alcohol, and the resultant precipitate was collected by filtration and washed with 500 parts of methyl alcohol, and then dried at 100° C. to obtain 24.1 parts of N,N,N′,N′-tetrakis{p-(cyclohexylmethyl-n-propyl)aminophenyl}-p-phenylene diamine.

To 24.1 parts of the obtained N,N,N′,N′-tetrakis{p-(cyclohexylmethyl-n-propyl)aminophenyl}-p-phenylene diamine were added 200 parts of acetonitrile and 7.9 parts of silver hexafluoro phosphate, and the resultant mixture was subjected to reaction at 60° C. for 3 hours, and the resultant silver was filtered off. Then, 200 parts of water was added to the filtrate, and the resultant precipitate was collected by filtration and dried to obtain 27.0 parts of N,N,N′,N′-tetrakis{p-(cyclohexylmethyl-n-propyl)aminophenyl}-p-phenylene diimmonium hexafluoro phosphate.

### Production Example 8

### Production of N,N,N′,N′-tetrakis{p-(cyclohexylmethyl-n-butyl)aminophenyl}-p-phenylene diimmonium hexafluoro phosphate

28.0 parts of N,N,N′,N′-tetrakis{p-(cyclohexyl methyl-n-butyl)aminophenyl}-p-phenylene diimmonium hexafluoro phosphate were obtained in substantially the same manner as in Production Example 7, except that instead of 1-iodo propane, 1-iodo butane was used.

### Production Example 9

### Production of N,N,N′,N′-tetrakis{p-di(cyclohexylmethyl)aminophenyl}-p-phenyl ene diimmonium bis(fluoro sulfonyl) imidate

27.0 parts of N,N,N′,N′-tetrakis{p-di(cyclohexylmethyl)aminophenyl}-p-phenyl ene diimmonium bis(fluoro sulfonyl) imidate were obtained in substantially the same manner as in Production Example 1, except that instead of silver hexafluoro phosphate, silver bis(fluoro sulfonyl) imidate was used.

### Example 1

In an automatic mortar AMN-200 (product of Nitto Kagaku Co., Ltd.), the N,N,N′,N′-tetrakis{p-di(cyclohexylmethyl)aminophenyl}-p-phenyl ene diimmonium hexafluoro phosphate obtained in Production Example 1 was placed, and then subjected to dry pulverizing with rotation velocity of 100 rpm for the mortar rod and rotation velocity of 6 rpm for the mortar container for about 30 minutes to form a pulverized product. The resultant pulverized product was measured on an X-ray diffraction device (RINT2200 manufactured by Rigaku Corporation) using CuKα ray as the X-ray source, with tube voltage of 40 kV, tube current of 20 mA, scanning range (2θ) in the range of 3-60°, spread slit of ½°, scattering slit of ½°, light receiving slit of 0.15 mm, sampling width of 0.02°, and scanning speed of 4°/min. The result of the measurement is shown in FIG. 1. The dye before dry pulverizing was also measured on the powder X-ray diffraction device in the same manner, and the result is shown in FIG. 2. The results indicate that the amorphous state is obtained through dry pulverizing. Specifically, the 6 sharp diffraction peaks near 15-25° in 2θ detected for the crystalline form become lower in intensity and broader in profile, and therefore no clear diffraction peaks can be observed. In each measurement, the full width at half maximum of the largest peak is measured, and it is 0.214 before dry pulverizing (FIG. 4), and 3.563 after dry pulverizing (FIG. 3). Then, 0.5 part of the pulverized dye and 9.5 parts of toluene were placed in a 50-mL glass vessel, and the mixture was agitated by means of a magnetic stirrer for 30 minutes, to obtain a mixture of diimmonium salt and toluene.

### Example 2

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(trifluoro methane sulfonyl) imidate obtained in Production Example 2 was used.

### Example 3)

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-propyl)aminophenyl}-p-phenylene diimmonium hexafluoro phosphate obtained in Production Example 3 was used.

### Example 4

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium hexafluoro antimonate obtained in Production Example 4 was used.

### Example 5

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-pentyl)aminophenyl}-p-phenylene diimmonium hexafluoro antimonate obtained in Production Example 5 was used.

### Example 6

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium bis(fluoro sulfonyl)imidate obtained in Production Example 6 was used.

### Example 7

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-propyl)aminophenyl}-p-phenylene diimmonium hexafluoro phosphate obtained in Production Example 7 was used.

### Example 8

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-butyl)aminophenyl}-p-phenylene diimmonium hexafluoro phosphate obtained in Production Example 8 was used.

### Example 9

A mixture of diimmonium salt and toluene was obtained in substantially the same manner as in Example 1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(fluoro sulfonyl) imidate obtained in Production Example 9 was used.

### Comparative Example 1-1

0.5 part of the N,N,N′,N′-tetrakis{p-di(cyclohexylmethyl)aminophenyl}-p-phenyl ene diimmonium hexafluoro phosphate obtained in Production Example 1 used as it is without dry pulverizing, as well as 9.5 parts of toluene, were placed in a 50-mL glass vessel, and mixed by means of a magnetic stirrer for 30 minutes to obtain a mixture of diimmonium salt and toluene.

### Comparative Example 1-2

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(trifluoromethane sulfonyl) imidate obtained in Production Example 2 was used.

### Comparative Example 1-3

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-propyl)aminophenyl}-p-phenylene diimmonium hexafluoro phosphate obtained in Production Example 3 was used.

### Comparative Example 1-4

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium hexafluoro antimonate obtained in Production Example 4 was used.

### Comparative Example 1-5

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-pentyl)aminophenyl}-p-phenylene diimmonium hexafluoro antimonate obtained in Production Example 5 was used.

### Comparative Example 1-6

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium bis(fluoro sulfonyl) imidate obtained in Production Example 6 was used.

### Comparative Example 1-7

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-propyl)aminophenyl}-p-phenylene diimmonium hexafluoro phosphate obtained in Production Example 7 was used.

### Comparative Example 1-8

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-butyl)aminophenyl}-p-phenylene diimmonium hexafluoro phosphate obtained in Production Example 8 was used.

### Comparative Example 1-9

A mixture of diimmonium salt and toluene was obtained in substantially the same manner as in Comparative Example 1-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(fluoro sulfonyl) imidate obtained in Production Example 9 was used.

### Comparative Example 2-1

0.5 part of the N,N,N′,N′-tetrakis{p-di(cyclohexylmethyl)aminophenyl}-p-phenyl ene diimmonium hexafluoro phosphate obtained in Production Example 1, 9.5 parts of toluene and 70 parts of zirconia beads with grain size of 0.3 mm were placed in a 50-mL glass vessel, and then subjected to wet pulverizing with shaking by means of a paint shaker for 2 hours to form a toluene wet dispersion liquid of diimmonium salt.

### Comparative Example 2-2

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(trifluoromethane sulfonyl) imidate obtained in Production Example 2 was used.

### Comparative Example 2-3

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-propyl)aminophenyl}-p-phenylene diimmonium hexafluoro phosphate obtained in Production Example 3 was used.

### Comparative Example 2-4

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium hexafluoro antimonate obtained in Production Example 4 was used.

### Comparative Example 2-5

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-pentyl)aminophenyl}-p-phenylene diimmonium hexafluoro antimonate obtained in Production Example 5 was used.

### Comparative Example 2-6

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(n-butyl)aminophenyl}-p-phenylene diimmonium bis(fluoro sulfonyl) imidate obtained in Production Example 6 was used.

### Comparative Example 2-7

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-propyl)aminophenyl}-p-phenylene diimmonium hexafluoro phosphate obtained in Production Example 7 was used.

### Comparative Example 2-8

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-(cyclohexylmethyl-n-butyl)aminophenyl}-p-phenylene diimmonium hexafluoro phosphate obtained in Production Example 8 was used.

### Comparative Example 2-9

A toluene wet dispersion liquid of diimmonium salt was obtained in substantially the same manner as in Comparative Example 2-1, except that instead of N,N,N′,N′-tetrakis{p-di(cyclohexylmethyl)aminophenyl}-p-phenyl ene diimmonium hexafluoro phosphate, the N,N,N′,N′-tetrakis{p-di(cyclohexylmethyl)aminophenyl}-p-phenyl ene diimmonium bis(fluoro sulfonyl) imidate obtained in Example 9 was used.

### Test Example 1

Each of the mixtures of diimmonium salt and toluene and the toluene wet dispersion liquid of diimmonium salt prepared in Example 1 and Comparative Examples 1-1 and 2-1 was filtered with a membrane filter with a 0.1 μm pore diameter to obtain a suspension. The obtained suspension was dried at 40° C. and under a reduced pressure of 10 Torr for 4 hours, followed by measurement on a powder X-ray diffraction device. The results of the measurement are shown in FIGS. 5-7. For each result, the full width at half maximum of the maximum peak was determined. It is 2.907 in Example 1 (FIG. 8), 0.214 in Comparative Example 1-1 (FIG. 9), and 0.366 in Comparative Example 2-1 (FIG. 10).

### Test Example 2

Using each of the mixtures of diimmonium salt and toluene and the toluene wet dispersion liquids of diimmonium salts prepared in Examples 1-9 and Comparative Examples 1-1 to 1-9 and 2-1 to 2-9, a glass sheet was evenly coated by means of a spin coater (1H-DX2, product of Mikasa Co., Ltd.) with a rotation velocity at 2000 rpm, followed by drying at 100° C. in a hot air circulating oven for 10 seconds. As a result, a film of the suspension was formed on the glass sheet. Then, the haze (turbidity) of the glass with film formed on it was measured by means of a haze meter NDH 5000 (product of Nippon Denshoku Industries Co., Ltd.). The result of the measurement is shown in Table 1.

### Test Example 3

1.1 parts of the mixture of diimmonium salt and toluene or 1.1 parts of the toluene wet dispersion liquid of diimmonium salt prepared in each of Examples 1-9 and Comparative Examples 1-1 to 1-9 and 2-1 to 2-9 were added to a solution containing 9.8 parts of acrylic-based adhesive SK DAIN 1811L (product of Soken Chemical & Engineering Co., Ltd.), 1.9 parts of toluene, 1.9 parts of ethyl acetate, and 0.02 part of curing agent TD-75 (product of Soken Chemical & Engineering Co., Ltd.) to form a near infrared absorptive adhesive composition. Using the obtained near infrared absorptive adhesive composition, a 25-μ-thick mold releasing film E7006 (product of Toyobo Co., Ltd.) was coated by means of a bar coater No. 60, followed by drying at 100° C. in a hot air circulating oven for 3 minutes. Then, a 50-μm-thick PET film A4300 (product of Toyobo Co., Ltd.) as a transparent substrate was bonded on the adhesive layer side, followed by curing at 40° C. for 2 days. The product was then bonded on glass to obtain a near infrared shielding filter.

For each near infrared shielding filter obtained as above, a heat resistance test was carried out by storing the sample in an atmosphere at 80° C. for 500 hours. After a prescribed time, transmissivity at wavelengths of 1000 nm and 550 nm was measured using a spectrophotometer, and the haze was measured using a haze meter. Also, a hygrothermal resistance test was carried out by storing each sample for 500 hours in an atmosphere at 60° C. and 95% RH. Then, the transmissivity at wavelengths of 1000 nm and 550 nm and the haze were measured in the same manner as in the heat resistance test. The results of the measurements are shown in Tables 2 and 3.

TABLE 1

| | | Comparative | | Comparative | |
|---|---|---|---|---|---|
| Example | Haze | Example 1 | Haze | Example 2 | Haze |
| 1 | 2.05 | 1 | 31.00 | 1 | 6.32 |
| 2 | 2.11 | 2 | 32.35 | 2 | 7.34 |
| 3 | 3.12 | 3 | 33.37 | 3 | 9.32 |
| 4 | 2.41 | 4 | 36.47 | 4 | 7.21 |
| 5 | 3.01 | 5 | 39.39 | 5 | 9.79 |
| 6 | 2.20 | 6 | 38.59 | 6 | 7.98 |
| 7 | 1.95 | 7 | 30.25 | 7 | 5.78 |
| 8 | 1.85 | 8 | 30.55 | 8 | 5.59 |
| 9 | 2.09 | 9 | 32.88 | 9 | 6.52 |

TABLE 2

| | Transmissivity | | | Transmissivity | | | | |
|---|---|---|---|---|---|---|---|---|
| | at 1000 nm (%) | | | at 550 nm (%) | | | Haze | |
| | | After | | | After | | | After |
| | Initial | test | [symbol] | Initial | test | [symbol] | Initial | test |
| Example | | | | | | | | |
| 1 | 2.5 | 3.0 | 0.5 | 80.3 | 80.2 | −0.1 | 1.87 | 1.85 |
| 2 | 4.3 | 5.0 | 0.7 | 80.9 | 80.1 | −0.8 | 1.88 | 1.90 |
| 3 | 6.8 | 7.7 | 0.9 | 77.4 | 76.9 | −0.5 | 2.02 | 2.11 |
| 4 | 6.3 | 7.2 | 0.9 | 78.1 | 76.3 | −1.8 | 1.86 | 1.90 |
| 5 | 7.0 | 8.0 | 1.0 | 78.4 | 75.1 | −3.3 | 2.17 | 2.10 |
| 6 | 6.9 | 7.5 | 0.6 | 77.9 | 77.1 | −0.9 | 2.20 | 2.23 |
| 7 | 3.1 | 4.0 | 0.9 | 79.4 | 77.0 | −2.4 | 1.79 | 1.81 |
| 8 | 3.5 | 4.2 | 0.7 | 79.2 | 77.7 | −1.5 | 1.79 | 1.82 |
| 9 | 2.8 | 3.5 | 0.7 | 80.2 | 79.6 | −0.6 | 1.89 | 1.88 |
| Comparative | | | | | | | | |
| Example 1 | | | | | | | | |
| 1 | 50.1 | 50.4 | 0.3 | 55.3 | 55.4 | 0.1 | 20.31 | 20.41 |
| 2 | 50.0 | 50.2 | 0.2 | 55.2 | 55.1 | −0.1 | 22.21 | 22.21 |
| 3 | 50.4 | 50.4 | 0.0 | 56.1 | 56.3 | 0.2 | 21.21 | 21.12 |
| 4 | 51.1 | 51.3 | 0.2 | 55.9 | 55.6 | −0.3 | 20.04 | 20.08 |
| 5 | 51.5 | 52.3 | 0.8 | 55.3 | 54.1 | −1.2 | 19.21 | 20.21 |
| 6 | 50.9 | 51.3 | 0.4 | 55.5 | 55.5 | 0.0 | 18.77 | 19.56 |
| 7 | 51.0 | 51.6 | 0.6 | 56.9 | 57.0 | 0.1 | 20.43 | 21.01 |
| 8 | 51.9 | 52.8 | 0.9 | 57.0 | 56.4 | −0.6 | 21.08 | 21.05 |
| 9 | 50.9 | 51.6 | 0.7 | 57.8 | 56.1 | −1.7 | 22.15 | 22.15 |
| Comparative | | | | | | | | |
| Example 2 | | | | | | | | |
| 1 | 6.2 | 11.0 | 4.8 | 68.7 | 62.4 | −6.3 | 3.42 | 4.01 |
| 2 | 15.4 | 21.2 | 5.8 | 64.5 | 60.3 | −4.2 | 4.31 | 5.59 |
| 3 | 10.8 | 16.4 | 5.6 | 65.3 | 60.4 | −4.9 | 4.62 | 5.97 |
| 4 | 10.5 | 15.2 | 4.7 | 70.3 | 65.1 | −5.2 | 5.67 | 6.98 |
| 5 | 23.1 | 29.3 | 6.2 | 70.4 | 65.1 | −5.3 | 6.09 | 7.68 |
| 6 | 10.5 | 16.5 | 6.0 | 68.8 | 60.4 | −8.4 | 4.31 | 4.01 |
| 7 | 8.5 | 13.9 | 5.4 | 69.6 | 63.2 | −6.4 | 3.72 | 4.29 |
| 8 | 9.3 | 14.2 | 4.9 | 70.4 | 65.1 | −5.3 | 3.52 | 3.71 |
| 9 | 9.8 | 13.5 | 3.7 | 69.4 | 64.1 | −5.3 | 3.50 | 4.00 |

* Results of test of heat resistance at 80° C.

TABLE 3

| | Transmissivity | | | Transmissivity | | | | |
|---|---|---|---|---|---|---|---|---|
| | at 1000 nm (%) | | | at 550 nm(%) | | | Haze | |
| | | After | | | After | | | After |
| | Initial | test | [symbol] | Initial | test | [symbol] | Initial | test |
| Example | | | | | | | | |
| 1 | 2.5 | 2.9 | 0.4 | 80.3 | 80.1 | −0.2 | 1.87 | 1.88 |
| 2 | 4.3 | 4.8 | 0.5 | 80.9 | 80.7 | −0.2 | 1.88 | 1.88 |
| 3 | 6.8 | 7.7 | 0.9 | 77.4 | 76.7 | −0.3 | 2.02 | 2.08 |
| 4 | 6.3 | 7.1 | 0.9 | 78.1 | 77.1 | −1.0 | 1.86 | 1.91 |
| 5 | 7.0 | 7.9 | 0.9 | 78.4 | 76.1 | −2.3 | 2.17 | 2.18 |
| 6 | 6.9 | 7.5 | 0.6 | 77.9 | 77.5 | −0.4 | 2.20 | 2.20 |
| 7 | 3.1 | 3.7 | 0.6 | 79.4 | 78.2 | −1.2 | 1.79 | 1.86 |
| 8 | 3.5 | 4.0 | 0.5 | 79.2 | 77.9 | −1.3 | 1.79 | 1.82 |
| 9 | 2.8 | 3.1 | 0.3 | 80.2 | 80.0 | −0.2 | 1.89 | 1.91 |
| Comparative | | | | | | | | |
| Example 1 | | | | | | | | |
| 1 | 50.1 | 50.4 | 0.3 | 55.3 | 55.3 | 0.0 | 20.31 | 20.32 |
| 2 | 50.0 | 50.1 | 0.1 | 55.2 | 55.2 | 0.0 | 22.21 | 22.20 |
| 3 | 50.4 | 50.6 | 0.2 | 56.1 | 56.3 | 0.2 | 21.21 | 21.21 |
| 4 | 51.1 | 51.2 | 0.1 | 55.9 | 55.5 | −0.4 | 20.04 | 20.04 |
| 5 | 51.5 | 51.9 | 0.4 | 55.3 | 54.8 | −0.5 | 19.21 | 19.22 |
| 6 | 50.9 | 51.0 | 0.1 | 55.5 | 55.2 | −0.3 | 18.77 | 20.54 |
| 7 | 51.0 | 51.4 | 0.4 | 56.9 | 57.2 | 0.3 | 20.43 | 20.21 |
| 8 | 51.9 | 52.7 | 0.8 | 57.0 | 57.4 | 0.4 | 21.08 | 22.54 |
| 9 | 50.9 | 51.1 | 0.2 | 57.8 | 57.4 | −0.4 | 22.15 | 23.18 |
| Comparative | | | | | | | | |
| Example 2 | | | | | | | | |
| 1 | 6.2 | 9.8 | 3.6 | 68.7 | 65.1 | −3.6 | 3.42 | 3.93 |
| 2 | 15.4 | 20.5 | 4.9 | 64.5 | 61.3 | −3.2 | 4.31 | 5.28 |
| 3 | 10.8 | 15.0 | 4.2 | 65.3 | 62.9 | −2.4 | 4.62 | 5.71 |
| 4 | 10.5 | 13.6 | 3.1 | 70.3 | 68.1 | −2.2 | 5.67 | 6.77 |
| 5 | 23.1 | 28.7 | 5.6 | 70.4 | 67.2 | −3.2 | 6.09 | 7.19 |
| 6 | 10.5 | 12.9 | 2.4 | 68.8 | 65.1 | −3.7 | 4.31 | 4.54 |
| 7 | 8.5 | 10.9 | 2.4 | 69.6 | 66.6 | −3.0 | 3.72 | 3.67 |
| 8 | 9.3 | 11.9 | 2.6 | 70.4 | 66.9 | −3.5 | 3.52 | 3.49 |
| 9 | 9.8 | 12.9 | 2.1 | 69.4 | 66.1 | −3.3 | 3.50 | 4.21 |

* Results of test of hygrothermal resistance at 60° C. and 95% RH.

In Table 1, a comparison of Example and Comparative Example 1 shows that the haze is lower in the amorphous form than in the crystal form. Also, a comparison of Example and Comparative Example 2 shows that even wet pulverizing is still insufficient to reduce the haze, yet the haze is significantly lower in the amorphous form.

As seen from Tables 2 and 3, in Comparative Example 1, the haze is high, and the near infrared absorptivity is considerably poor. In Comparative Example 2, although the near infrared absorptivity is improved compared with Comparative Example 1, the haze is still high because of the crystallinity of the contained diimmonium salt. Also in Comparative Example 2, coagulation of the dye particles takes place over the process of the test, leading to a rise in the haze and degradation of the near infrared absorptivity.

The results show that by using the amorphous form of diimmonium salt, it is possible to achieve a low haze and excellent transparency as well as high heat resistance and hygrothermal resistance.

### Test Example 4

14 parts of the mixture of diimmonium salt and toluene or 14 parts of the toluene wet dispersion liquid of diimmonium salt prepared in each of Examples 1, 6, 8, 9 and Comparative Examples 1-1, 1-6, 1-8, 1-9 and Comparative Examples 2-1, 2-6, 2-8, 2-9 were added to a solution containing 28 parts of UV curable hard coat agent UN-3320HC (product of Negami Chemical Industrial Co., Ltd.) with urethane acrylate resin as the principal ingredient, 28 parts of methyl isobutyl ketone, and 28 parts of toluene. Then, IRGACURE 184 (product of Ciba Specialty Corp.), a photo-polymerization initiator, was added to form a resin composition for hard coat. Using the obtained resin composition for hard coat, a 100-μm-thick PET film (A4300, product of Toyobo Co., Ltd.) as a transparent substrate was coated by means of a bar coater No. 12, followed by drying at 100° C. for 1 minute. Then, UV light with an intensity of 60 mJ/cm² was irradiated for polymerizing and curing the coated film. As a result, a near infrared shielding filter was obtained.

The obtained near infrared shielding filter was subjected to the heat resistance and the hygrothermal resistance test in the same way as in Test Example 3 to measure the transmissivity at wavelengths of 1000 nm and 550 nm, and the haze. The results of the measurement are shown in Tables 4 and 5.

TABLE 4

| | Transmissivity | | | Transmissivity | | | | |
|---|---|---|---|---|---|---|---|---|
| | at 1000 nm (%) | | | at 550 nm (%) | | | Haze | |
| | | After | | | After | | | After |
| | Initial | test | [symbol] | Initial | test | [symbol] | Initial | test |
| Example | | | | | | | | |
| 1 | 9.5 | 9.8 | 0.3 | 87.6 | 87.1 | −0.5 | 2.05 | 2.12 |
| 6 | 11.5 | 12.0 | 0.5 | 84.5 | 84.1 | −0.4 | 2.15 | 2.23 |
| 8 | 10.3 | 10.9 | 0.7 | 86.4 | 85.7 | −0.7 | 1.91 | 1.95 |
| 9 | 10.5 | 10.5 | 0.0 | 87.9 | 87.4 | −0.5 | 2.25 | 2.31 |
| Comparative | | | | | | | | |
| Example 1 | | | | | | | | |
| 1 | 54.6 | 54.7 | 0.1 | 59.9 | 59.5 | −0.4 | 17.44 | 17.59 |
| 6 | 55.8 | 56.1 | 0.3 | 60.1 | 60.5 | 0.4 | 18.30 | 18.29 |
| 8 | 53.5 | 54.3 | 0.8 | 58.3 | 57.9 | −0.4 | 16.43 | 16.33 |
| 9 | 54.2 | 54.5 | 0.3 | 60.5 | 60.2 | −0.3 | 18.20 | 18.51 |
| Comparative | | | | | | | | |
| Example 2 | | | | | | | | |
| 1 | 10.5 | 11.2 | 0.7 | 86.6 | 85.8 | −0.8 | 2.49 | 2.74 |
| 6 | 13.6 | 14.8 | 1.2 | 79.5 | 78.9 | −0.6 | 3.02 | 3.11 |
| 8 | 11.7 | 13.5 | 1.8 | 83.1 | 81.5 | −1.6 | 2.85 | 2.99 |
| 9 | 12.4 | 13.9 | 1.5 | 84.6 | 82.5 | −2.1 | 2.68 | 3.01 |

* Results of test of heat resistance at 80° C.

TABLE 5

| | Transmissivity | | | Transmissivity | | | | |
|---|---|---|---|---|---|---|---|---|
| | at 1000 nm (%) | | | at 550 nm (%) | | | Haze | |
| | | After | | | After | | | After |
| | Initial | test | [symbol] | Initial | test | [symbol] | Initial | test |
| Example | | | | | | | | |
| 1 | 9.5 | 9.9 | 0.4 | 87.6 | 86.1 | −1.5 | 2.05 | 2.21 |
| 6 | 11.5 | 12.2 | 0.7 | 84.5 | 83.9 | −0.6 | 2.15 | 2.20 |
| 8 | 10.3 | 11.7 | 1.4 | 86.4 | 84.7 | −1.7 | 1.91 | 2.04 |
| 9 | 10.5 | 10.8 | 0.3 | 87.9 | 87.7 | −0.2 | 2.25 | 2.50 |
| Comparative | | | | | | | | |
| Example 1 | | | | | | | | |
| 1 | 54.6 | 54.9 | 0.5 | 59.9 | 59.3 | −0.5 | 17.44 | 18.54 |
| 6 | 55.8 | 56.1 | 0.3 | 60.1 | 59.5 | −0.6 | 18.30 | 18.80 |
| 8 | 53.5 | 55.0 | 1.5 | 58.3 | 57.1 | −1.2 | 16.43 | 17.82 |
| 9 | 54.2 | 54.2 | 0.0 | 60.5 | 59.8 | −0.7 | 18.2 | 19.23 |
| Comparative | | | | | | | | |
| Example 2 | | | | | | | | |
| 1 | 10.5 | 12.9 | 1.4 | 86.6 | 86.0 | −0.6 | 2.49 | 2.58 |
| 6 | 13.6 | 14.2 | 0.6 | 79.5 | 78.7 | −0.8 | 3.02 | 3.18 |
| 8 | 11.7 | 14.7 | 3.0 | 83.1 | 80.2 | −2.9 | 2.85 | 3.35 |
| 9 | 12.4 | 14.1 | 1.7 | 84.6 | 83.3 | −1.3 | 2.68 | 3.41 |

* Results of test of hygrothermal resistance at 60° C. and 95% RH.

As is the case for the results shown in Tables 2 and 3, it can be seen from Tables 4 and 5 that the haze is high, and the near infrared absorptivity is considerably poor in Comparative Example 1. In Comparative Example 2, although the near infrared absorptivity is improved compared with Comparative Example 1, the haze is still high because of the crystallinity of the contained diimmonium salt. Also in Comparative Example 2, coagulation of the dye particles takes place over the process of the test, leading to a rise in the haze and degradation of the near infrared absorptivity.

The results show that by using the amorphous form of diimmonium salt, it is possible to achieve a low haze and excellent transparency as well as high heat resistance and high hygrothermal resistance.

### BRIEF DESCRIPTION OF THE DRAWINGS

FIG. 1 is a diagram illustrating the results of the X-ray diffraction after dry pulverizing of the sample prepared in Production Example 1.

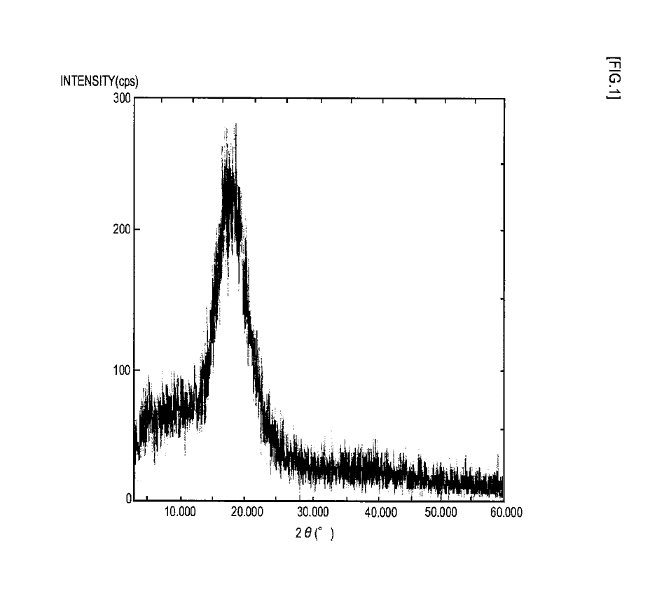

FIG. 2 is a diagram illustrating the results of the X-ray diffraction before pulverizing of the sample prepared in Production Example 1.

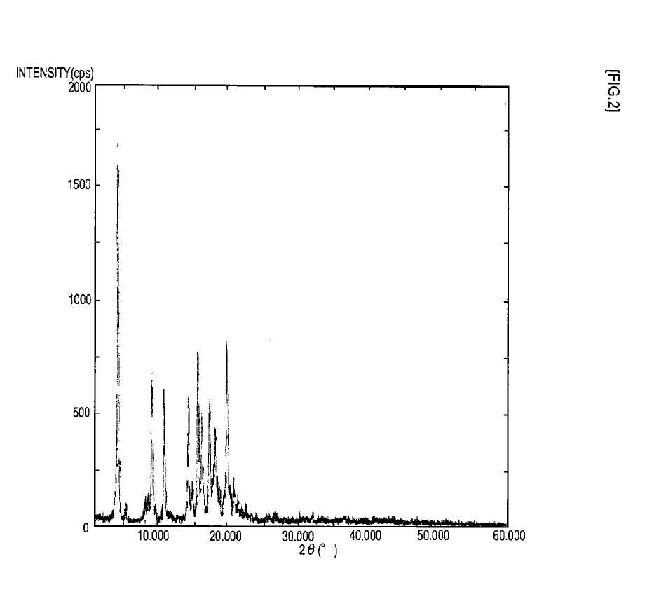

FIG. 3 is a diagram illustrating the full width at half maximum of the maximum peak in the results of the X-ray diffraction for the sample of Production Example 1 after dry pulverizing.

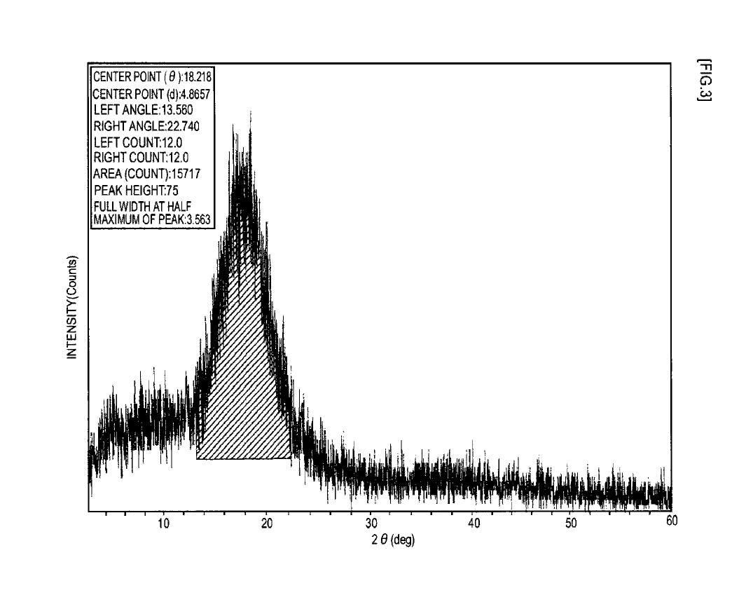

FIG. 4 is a diagram illustrating the full width at half maximum of the maximum peak in the results of the X-ray diffraction for the sample Production Example 1 of before pulverizing.

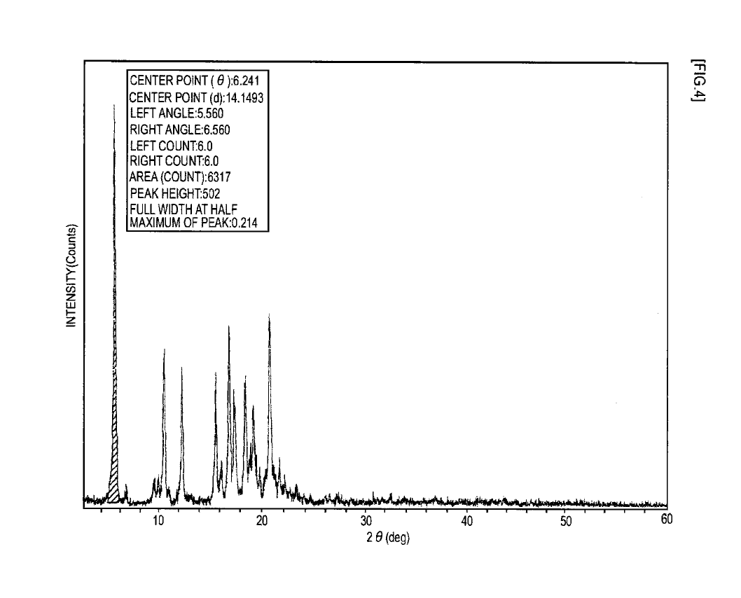

FIG. 5 is a diagram illustrating the results of the X-ray diffraction for Example 1 in Test Example 1.

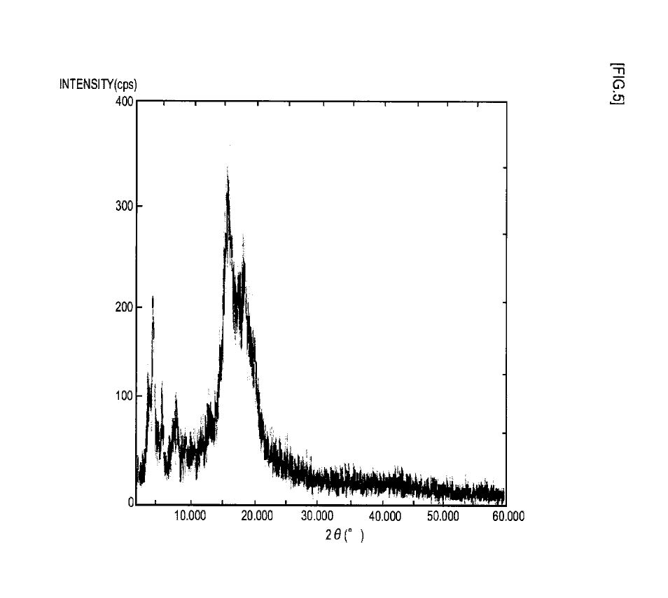

FIG. 6 is a diagram illustrating the results of the X-ray diffraction for Comparative Example 1-1 in Test Example 1.

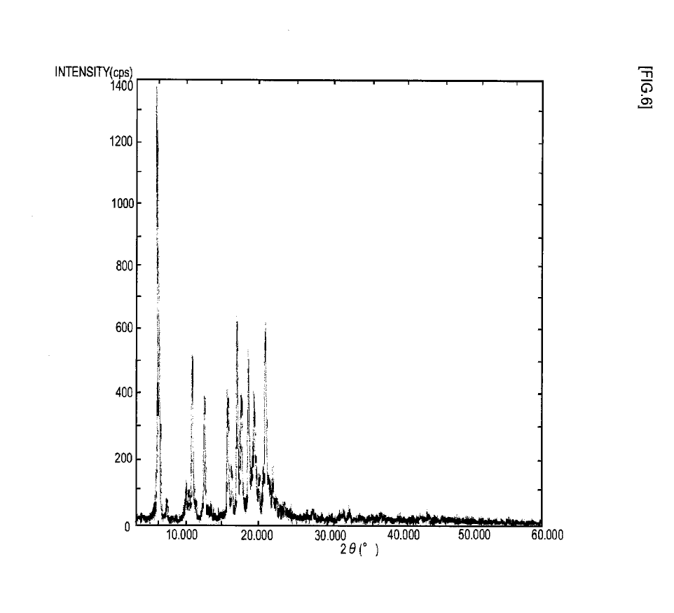

FIG. 7 is a diagram illustrating the results of the X-ray diffraction for Comparative Example 2-1 in Test Example 1.

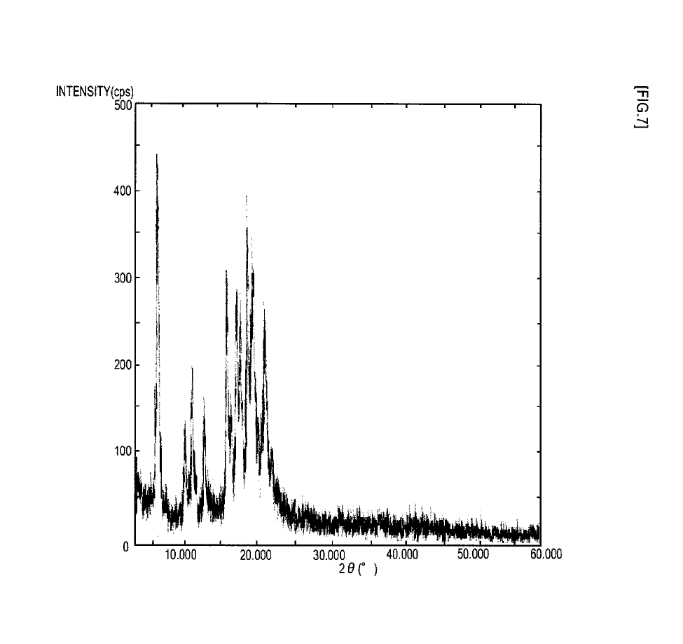

FIG. 8 is a diagram illustrating the full width at half maximum of the maximum peak in the results of the X-ray diffraction for the sample of Example 1 in Test Example 1.

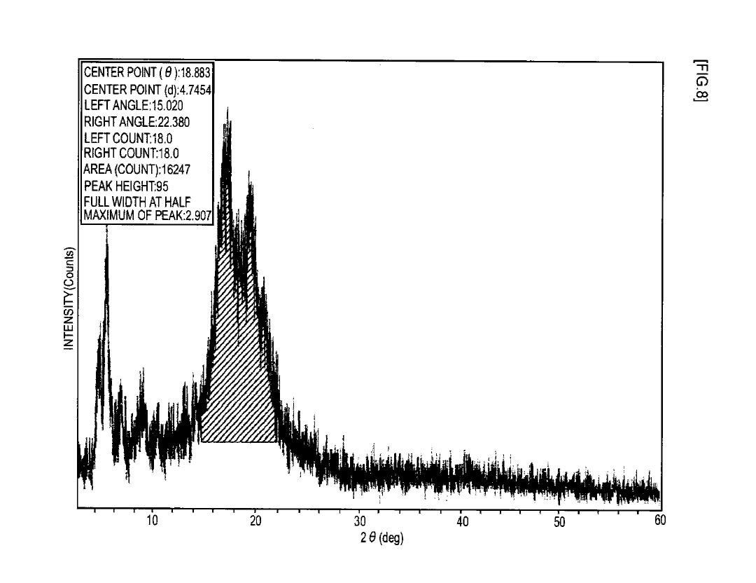

FIG. 9 is a diagram illustrating the full width at half maximum of the maximum peak in the results of the X-ray diffraction for the sample of Comparative Example 1-1 in Test Example 1.

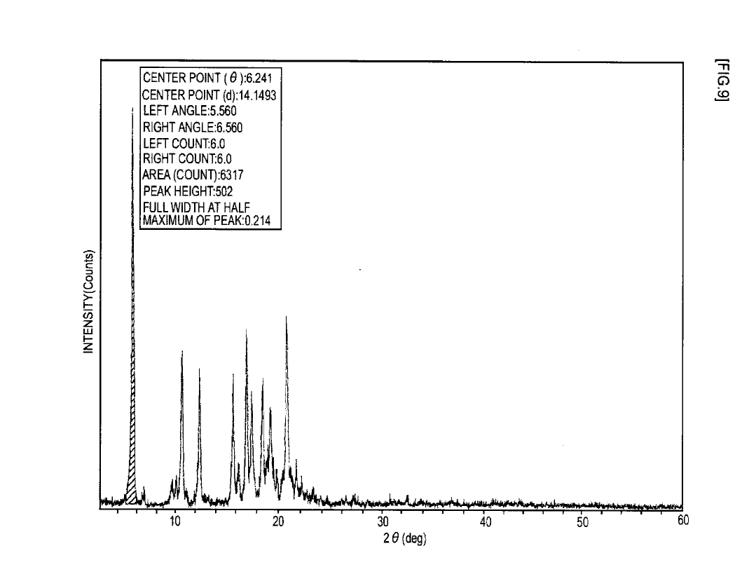

FIG. 10 is a diagram illustrating the full width at half maximum of the maximum peak in the results of the X-ray diffraction for the sample of Comparative Example 2-1 in Test Example 1.

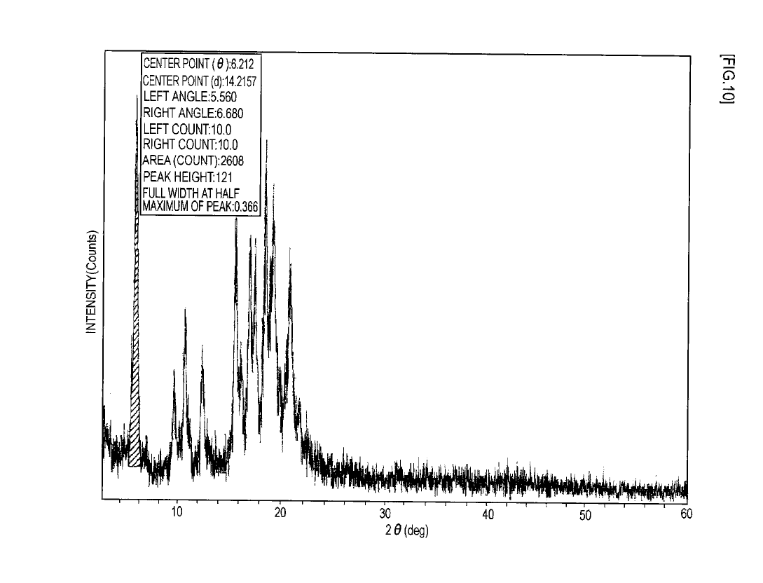

### INDUSTRIAL APPLICABILITY

The near infrared absorptive composition prepared using the near infrared absorbent dye made of the amorphous form of diimmonium salt of the present invention has high heat resistance and hygrothermal resistance as well as excellent transparency, with no decrease in the near infrared absorptivity over a long period of time. Consequently, it can be used in various applications, such as PDP, automobile glass, and building glass.

1. A near infrared absorbent dye, comprising:
an amorphous diimmonium salt of formula (1): 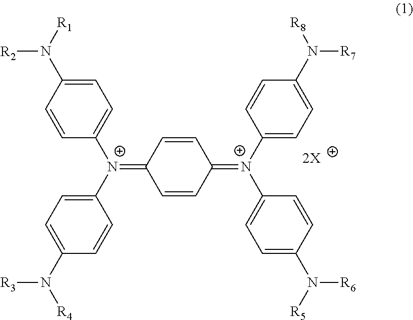
wherein each of R₁ to R₈ is independently an organic group, and
X is an anion.

2. The dye of claim 1,
wherein the organic groups R₁ to R₈ are identical and are selected from the group consisting of an n-propyl group, an n-butyl group, an n-pentyl group, an n-hexyl group, and a cyclohexyl methyl group.

3. The dye of claim 1,
wherein the organic groups R₁ to R₈ are not all identical and are each independently selected from the group consisting of an n-propyl group, an n-butyl group, an n-pentyl group, an n-hexyl group, and a cyclohexyl methyl group.

4. The dye of claim 1,
wherein the organic groups R₁ to R₈ collectively consist of two different types of organic groups:
at least one cyclohexyl methyl group, and
at least one organic group selected from the group consisting of an n-propyl group, an n-butyl group, an n-pentyl group, and an n-hexyl group.

5. The dye of claim 4,
wherein R₁ and R₂ are different,
R₃ and R₄ are different,
R₅ and R₆ are different, and
R₇ and R₈ are different.

6. The dye of claim 1,
wherein X⁻ is selected from the group consisting of a hexafluoro phosphate ion, a tetrafluoro borate ion, a hexafluoro antimonate ion, a bis(trifluoromethane sulfonyl) imidate ion, and a bis(fluorosulfonyl) imidate ion.

7. The dye of claim 1,
wherein the amorphous diimmonium salt is obtained by a process comprising dry pulverizing a crystalline solid of the diimmonium salt.

8. A near infrared absorptive adhesive composition, comprising:
an adhesive, comprising the dye of claim 1 in a solid state.

9. A near infrared shielding filter, comprising:
an adherence layer comprising the near infrared absorptive adhesive composition of claim 8.

10. A near infrared absorptive resin composition comprising:
an active energy radiation curable resin, comprising the dye of claim 1 in a solid state,
wherein the near infrared absorptive resin composition is suitable for hard coat.

11. The resin composition of claim 10, wherein the active energy radiation curable resin is at least one resin selected from the group consisting of a polyester-based resin, an acrylic-based resin, a polyamide-based resin, a polyurethane-based resin, and a polyolefin-based resin.

12. A near infrared absorptive hard coat material, comprising:
a hard coat layer obtained by a process comprising irradiating the resin composition of claim 10, thereby curing the resin composition.

13. The near infrared absorptive hard coat material of claim 12, wherein the hard coat layer is on a surface of a transparent substrate.

14. The near infrared absorptive hard coat material of claim 13, wherein the transparent substrate is at least one substrate selected from the group consisting of glass, PET film, TAC film, and electromagnetic wave shielding film.

15. A near infrared shielding filter, comprising the near infrared absorptive hard coat material of claim 12.

16. The adhesive composition of claim 8, further comprising a solvent.

17. The adhesive composition of claim 16, wherein a boiling point of the solvent is 200° C. or lower.

18. The adhesive composition of claim 16, wherein a water content of the solvent is 5 mass % or less.

19. A method of manufacturing a near infrared absorptive adhesive composition, comprising:
manufacturing the adhesive composition with the dye of claim 1 in a solid state.

20. A method of manufacturing a near infrared absorptive hard coat material, the method comprising:
irradiating the resin composition of claim 10, thereby curing the resin composition.
